(19) United States
(12) Patent Application Publication
(10) Pub. No.: US 2023/0287478 A1
(43) Pub. Date: Sep. 14, 2023

(54) CONCATEMERIC DETECTABLE PROBES AND RELATED METHODS

(71) Applicant: 10x Genomics, Inc., Pleasanton, CA (US)

(72) Inventor: Felice Alessio BAVA, Rome (IT)

(73) Assignee: 10x Genomics, Inc., Pleasanton, CA (US)

(21) Appl. No.: 18/120,024

(22) Filed: Mar. 10, 2023

Related U.S. Application Data
(60) Provisional application No. 63/319,168, filed on Mar. 11, 2022.

(51) Int. Cl. C12Q 1/6816; C12Q 1/6844; C12Q 1/6874

(52) U.S. Cl. CPC C12Q 1/6816; C12Q 1/6844; C12Q 1/6874

(57) ABSTRACT

The present disclosure relates in some aspects to methods, probes, and kits for detection of a target analyte in a sample. In some embodiments, disclosed herein are methods in which a biological sample is contacted with a pre-formed detectable probe comprising a concatemeric region, e.g., a rolling circle amplification (RCA) product, comprising multiple copies of a unit sequence. Also disclosed herein are related probes and kits.

### CROSS-REFERENCE TO RELATED APPLICATION

This application claims priority to U.S. Provisional Patent Application No. 63/319,168, filed Mar. 11, 2022, entitled “CONCATEMERIC DETECTABLE PROBES AND RELATED METHODS,” which is herein incorporated by reference in its entirety for all purposes.

### FIELD

The present disclosure relates in some aspects to methods, probes, and kits for analysis or detection of analytes in a biological sample, particularly for in situ analysis of biomolecules in a cell or tissue sample.

### BACKGROUND

Methods are available for analyzing nucleic acids present in a biological sample, such as a cell or a tissue sample. Current methods for analyzing nucleic acids present in a biological sample, for example for in situ analysis, can have low sensitivity and specificity, have limited plexity, or be biased, time-consuming, labor-intensive, and/or error-prone. Improved methods for analyzing nucleic acids present in a biological sample are needed. Provided herein are methods, probes, and kits that meet such and other needs.

### SUMMARY

In some embodiments, provided herein is a method for analyzing a biological sample, comprising: (a) contacting the biological sample with a pre-formed detectable probe comprising (i) a target-binding region that hybridizes to a target sequence and (ii) a concatemeric region comprising at least 20 copies of a unit sequence, wherein the biological sample comprises a target nucleic acid comprising the target sequence; and (b) detecting a signal associated with the detectable probe, thereby detecting the target nucleic acid or a sequence thereof in the biological sample.

In any of the embodiments herein, the concatemeric region of the pre-formed detectable probe can be a rolling circle amplification (RCA) product generated prior to the contacting in step (a).

In some embodiments, provided herein is a method for analyzing a biological sample, comprising: (a) contacting the biological sample with a detectable probe, wherein the biological sample comprises a target nucleic acid comprising a target sequence, and wherein the detectable probe comprises (i) a target-binding region that hybridizes to the target sequence and (ii) a concatemeric region comprising multiple copies of a unit sequence, wherein the concatemeric region is a rolling circle amplification (RCA) product generated prior to contacting the biological sample; and (b) detecting a signal associated with the detectable probe, thereby detecting the target nucleic acid or a sequence thereof in the biological sample.

In any of the embodiments herein, the detectable probe can comprise a detectable label. In any of the embodiments herein, the detectable probe can comprise one or more detectably labelled nucleotides. In any of the embodiments herein, the one or more detectably labelled nucleotides can be incorporated into the concatemeric region during RCA.

In any of the embodiments herein, the unit sequence can be complementary to a circular or circularized oligonucleotide amplified by rolling circle amplification (RCA) to generate the concatemeric region of the detectable probe. In any of the embodiments herein, the unit sequence can comprise a detection region capable of binding directly or indirectly to a detectably labeled oligonucleotide. In any of the embodiments herein, the unit sequence can comprise a detection region capable of binding directly to a detectably labeled oligonucleotide. In any of the embodiments herein, the detection region can comprise all or a portion of the unit sequence. In any of the embodiments herein, the detection region can be the unit sequence.

In any of the embodiments herein, the unit sequence can comprise a barcode region. In any of the embodiments herein, the barcode region in the unit sequence can comprise one or more barcode sequences corresponding to the target nucleic acid.

In any of the embodiments herein, the method can further comprise, before the detecting in step (b), hybridizing the detectably labeled oligonucleotide to the detectable probe or to an intermediate probe that hybridizes to the detectable probe. In any of the embodiments herein, the hybridizing to the intermediate probe can thereby indirectly hybridize the detectably labeled oligonucleotide to the detectable probe. In any of the embodiments herein, the detectable probe or the intermediate probe can be contacted with, e.g., hybridized to, the detectably labeled oligonucleotide before the contacting in step (a), such that the detectably labeled oligonucleotide is pre-bound to the detectable probe or the intermediate probe before the detectably labeled oligonucleotide contacts the biological sample. In any of the embodiments herein, the detectable probe or the intermediate probe can be contacted with, e.g., hybridized to, the detectably labeled oligonucleotide after the contacting in step (a), such that the detectably labeled oligonucleotide contacts the biological sample after the detectable probe or the intermediate probe contacts the biological sample.

In any of the embodiments herein, the detecting in step (b) can comprise detecting the detectably labeled oligonucleotide. In any of the embodiments herein, the detecting in step (b) can comprise detecting a complex comprising multiple molecules of the detectably labeled oligonucleotide.

In any of the embodiments herein, the detecting in step (b) can be performed in situ in the biological sample.

In any of the embodiments herein, the concatemeric region can comprise between about 20 and about 1,000 copies of the unit sequence. In any of the embodiments herein, the concatemeric region can comprise between about 50 or between about 200 copies of the unit sequence.

In any of the embodiments herein, the target-binding region may be non-overlapping with the concatemeric region. In any of the embodiments herein, the target-binding region can be within the concatemeric region. In any of the embodiments herein, the target-binding region can be within the unit sequence of the concatemeric region.

In any of the embodiments herein, in the detectable probe, the copy number of the detection region and the copy number of the target-binding region can be at a ratio of between about 1,000:1 and about 1:1. In any of the embodiments herein, in the detectable probe, the copy number of the detection region and the copy number of the target-binding region can be at a ratio of between about 100:1 and about 1:1.

In any of the embodiments herein, the detectable probe can comprise one or more cleavage sites, optionally wherein the one or more cleavage sites can be cleavable enzymatically and/or chemically. In any of the embodiments herein, the one or more cleavage sites can be cleavable enzymatically and/or chemically. In any of the embodiments herein, the detectable probe can comprise one or more cleavage sites in the target-binding region, between the target-binding region and the concatemeric region, and/or in the concatemeric region. In any of the embodiments herein, the detectable probe can comprise one or more cleavage sites in the target-binding region. In any of the embodiments herein, the detectable probe can comprise one or more cleavage sites between the target-binding region and the concatemeric region. In any of the embodiments herein, the detectable probe can comprise one or more cleavage sites in the concatemeric region. In any of the embodiments herein, the one or more cleavage sites can be in the unit sequence of the concatemeric region.

In any of the embodiments herein, the concatemeric region can be a rolling circle amplification (RCA) product of a circular or circularized oligonucleotide. In any of the embodiments herein, the unit sequence of the concatemeric region can comprise a detection region, and the circular or circularized oligonucleotide can comprise a sequence complementary to the detection region. In any of the embodiments herein, the circular or circularized oligonucleotide can further comprise a sequence complementary to the target-binding region.

In any of the embodiments herein, the detectable probe can be generated prior to the contacting in step (a) by: (i) hybridizing a circular oligonucleotide or a circularizable oligonucleotide or oligonucleotide set to a binding oligonucleotide, and (ii) performing RCA using the circular oligonucleotide or a circularized oligonucleotide as template, wherein the circularized oligonucleotide is generated by circularizing the circularizable oligonucleotide or oligonucleotide set, and wherein the RCA is primed by the binding oligonucleotide or a product thereof

In any of the embodiments herein, the method can further comprise generating the detectable probe, wherein the generation can comprise: (i) hybridizing a circular oligonucleotide or a circularizable oligonucleotide or oligonucleotide set to a binding oligonucleotide, and (ii) performing a rolling circle amplification (RCA) using the circular oligonucleotide or a circularized oligonucleotide as template, wherein the circularized oligonucleotide can be generated by circularizing the circularizable oligonucleotide or oligonucleotide set, and wherein the RCA can be primed by the binding oligonucleotide or a product thereof.

In any of the embodiments herein, the binding oligonucleotide can comprise the target-binding region or a portion thereof. In any of the embodiments herein, the binding oligonucleotide can comprise a portion of the unit sequence.

In any of the embodiments herein, the unit sequence can comprise a detection region, and the binding oligonucleotide can comprise the detection region or a portion thereof. In any of the embodiments herein, the circular or circularized oligonucleotide can comprise a sequence complementary to the detection region or portion thereof.

In any of the embodiments herein, the unit sequence can comprise the detection region, and the circular or circularized oligonucleotide can comprise a sequence complementary to the detection region or a portion thereof.

In any of the embodiments herein, the unit sequence can comprise a detection region, and the binding oligonucleotide may not comprise the detection region or a portion thereof.

In any of the embodiments herein, the circular or circularized oligonucleotide may not comprise a sequence complementary to the detection region or portion thereof. In any of the embodiments herein, the circular or circularized oligonucleotide can comprise a sequence complementary to the target-binding region or a portion thereof. In any of the embodiments herein, the circular or circularized oligonucleotide may not comprise a sequence complementary to the target-binding region or a portion thereof. In any of the embodiments herein, the circular or circularized oligonucleotide can comprise a sequence complementary to a sequence in the unit sequence other than the detection region.

In any of the embodiments herein, the RCA, e.g., for generating the detectable probe, can be performed for less than about 5 minutes, less than about 10 minutes, less than about 15 minutes, less than about 30 minutes, less than about one hour, less than about two hours, or less than about three hours. In any of the embodiments herein, the RCA, e.g., for generating the detectable probe, can be performed for less than about 15 minutes. In any of the embodiments herein, the RCA, e.g., for generating the detectable probe, can be performed for less than about 30 minutes. In any of the embodiments herein, the RCA, e.g., for generating the detectable probe, can be performed for less than about one hour. In any of the embodiments herein, the RCA, e.g., for generating the detectable probe, can be performed for less than about two hours. In any of the embodiments herein, the RCA, e.g., for generating the detectable probe, can be performed for less than about three hours.

In any of the embodiments herein, the concatemeric region can be in the form of a nanoball. In any of the embodiments herein, the concatemeric region can be in the form of a nanoball having a diameter of between about 0.1 μm and about 1.5 μm, between about 0.15 μm and about 1 μm, between about 0.2 μm and about 0.5 μm, or between about 0.3 μm and about 0.4 μm. In any of the embodiments herein, the concatemeric region can be in the form of a nanoball having a diameter of between about 0.1 μm and about 1.5 μm. In any of the embodiments herein, the concatemeric region can be in the form of a nanoball having a diameter of between about 0.15 μm and about 1 μm. In any of the embodiments herein, the concatemeric region can be in the form of a nanoball having a diameter of between about 0.2 μm and about 0.5 μm. In any of the embodiments herein, the concatemeric region can be in the form of a nanoball having a diameter of between about 0.3 μm and about 0.4 μm.

In any of the embodiments herein, the concatemeric region can be between about 1 and about 15 kilobases or between about 15 and about 25 kilobases in length. In any of the embodiments herein, the concatemeric region can be between about 1 and about 15 kilobases in length. In any of the embodiments herein, the concatemeric region can be between about 15 and about 25 kilobases in length.

In any of the embodiments herein, the detectable probe can be produced ex situ. In any of the embodiments herein, the detectable probe can be produced in vitro.

In any of the embodiments herein, the target nucleic acid can comprise or can be a cellular nucleic acid molecule. In any of the embodiments herein, the target nucleic acid can comprise or can be genomic DNA, mRNA, or cDNA. In any of the embodiments herein, the target nucleic acid can comprise or can be genomic DNA. In any of the embodiments herein, the target nucleic acid can comprise or can be mRNA. In any of the embodiments herein, the target nucleic acid can comprise or can be cDNA.

In any of the embodiments herein, the target nucleic acid can comprise or can be a primary probe that hybridizes to a cellular nucleic acid molecule. In any of the embodiments herein, the primary probe can be selected from the group consisting of: a primary probe comprising a 3′ or 5′ overhang upon hybridization to the cellular nucleic acid molecule, optionally wherein the 3′ or 5′ overhang comprises one or more barcode sequences; a primary probe comprising a 3′ overhang and a 5′ overhang upon hybridization to the cellular nucleic acid molecule, optionally wherein the 3′ overhang and the 5′ overhang each independently comprises one or more barcode sequences; a circular primary probe; a circularizable primary probe or probe set; a primary probe or probe set comprising a split hybridization region configured to hybridize to a splint, optionally wherein the split hybridization region comprises one or more barcode sequences; and any combination thereof. In any of the embodiments herein, the target-binding region of the detectable probe can hybridize to the one or more barcode sequences in the primary probe.

In any of the embodiments herein, the primary probe can be a primary probe comprising a 3′ or 5′ overhang upon hybridization to the cellular nucleic acid molecule. In any of the embodiments herein, the 3′ or 5′ overhang can comprise one or more barcode sequences.

In any of the embodiments herein, the primary probe can be a primary probe comprising a 3′ overhang and a 5′ overhang upon hybridization to the cellular nucleic acid molecule. In any of the embodiments herein, the 3′ overhang and the 5′ overhang can each independently comprise one or more barcode sequences.

In any of the embodiments herein, the primary probe can be a circular primary probe.

In any of the embodiments herein, the primary probe can be a circularizable primary probe or probe set.

In any of the embodiments herein, the primary probe can be a primary probe or probe set comprising a split hybridization region configured to hybridize to a splint. In any of the embodiments herein, the split hybridization region can comprise one or more barcode sequences.

In any of the embodiments herein, the primary probe can comprise one or more barcode sequences. In any of the embodiments herein, the target-binding region of the detectable probe can hybridize to the one or more barcode sequences in the primary probe.

In any of the embodiments herein, the target nucleic acid can comprise or can be an intermediate probe that hybridizes to a primary probe or a product or complex thereof, wherein the primary probe can hybridize to a cellular nucleic acid molecule. In any of the embodiments herein, the product or complex of the primary probe can be selected from the group consisting of: a rolling circle amplification (RCA) product, a complex comprising an initiator and an amplifier for hybridization chain reaction (HCR), a complex comprising an initiator and an amplifier for linear oligonucleotide hybridization chain reaction (LO-HCR), a primer exchange reaction (PER) product, and a complex comprising a pre-amplifier and an amplifier for branched DNA (bDNA). In any of the embodiments herein, the product or complex of the primary probe can be a rolling circle amplification (RCA) product. In any of the embodiments herein, the product or complex of the primary probe can be a complex comprising an initiator and an amplifier for hybridization chain reaction (HCR). In any of the embodiments herein, the product or complex of the primary probe can be a complex comprising an initiator and an amplifier for linear oligonucleotide hybridization chain reaction (LO-HCR). In any of the embodiments herein, the product or complex of the primary probe can be a primer exchange reaction (PER) product. In any of the embodiments herein, the product or complex of the primary probe can be a complex comprising a pre-amplifier and an amplifier for branched DNA (bDNA).

In any of the embodiments herein, the intermediate probe can be selected from the group consisting of: an intermediate probe comprising a 3′ or 5′ overhang upon hybridization to the primary probe or product or complex thereof, optionally wherein the 3′ or 5′ overhang comprises one or more barcode sequences; an intermediate probe comprising a 3′ overhang and a 5′ overhang upon hybridization to the primary probe or product or complex thereof, optionally wherein the 3′ overhang and the 5′ overhang each independently comprises one or more barcode sequences; a circular intermediate probe; a circularizable intermediate probe or probe set; an intermediate probe or probe set comprising a split hybridization region configured to hybridize to a splint, optionally wherein the split hybridization region comprises one or more barcode sequences; and any combination thereof. In any of the embodiments herein, the target-binding region of the detectable probe can hybridize to the one or more barcode sequences in the intermediate probe.

In any of the embodiments herein, the intermediate probe can be an intermediate probe comprising a 3′ or 5′ overhang upon hybridization to the primary probe or product or complex thereof. In any of the embodiments herein, the 3′ or 5′ overhang can comprise one or more barcode sequences.

In any of the embodiments herein, the intermediate probe can be an intermediate probe comprising a 3′ overhang and a 5′ overhang upon hybridization to the primary probe or product or complex thereof. In any of the embodiments herein, the 3′ overhang and the 5′ overhang can each independently comprise one or more barcode sequences.

In any of the embodiments herein, the intermediate probe can be a circular intermediate probe.

In any of the embodiments herein, the intermediate probe can be a circularizable intermediate probe or probe set.

In any of the embodiments herein, the intermediate probe can be an intermediate probe or probe set comprising a split hybridization region configured to hybridize to a splint. In some of any embodiments, the split hybridization region can comprise one or more barcode sequences.

In any of the embodiments herein, the intermediate probe can comprise one or more barcode sequences. In any of the embodiments herein, the target-binding region of the detectable probe can hybridize to the one or more barcode sequences in the intermediate probe.

In any of the embodiments herein, the target nucleic acid can comprise or can be a reporter oligonucleotide of a labeling agent, the labeling agent comprising an analyte-binding region and the reporter oligonucleotide, optionally wherein the analyte can comprise a nucleic acid, a protein, a carbohydrate, a lipid, or a small molecule, or a complex thereof. In any of the embodiments herein, the analyte can comprise a nucleic acid, a protein, a carbohydrate, a lipid, or a small molecule, or a complex thereof.

In any of the embodiments herein, the target nucleic acid can comprise an overhang region comprising multiple copies of the target sequence; and/or the target nucleic acid can be concatemeric and comprise multiple copies of the target sequence. In any of the embodiments herein, the target nucleic acid can comprise an overhang region comprising multiple copies of the target sequence. In any of the embodiments herein, the target nucleic acid can be concatemeric and comprise multiple copies of the target sequence.

In any of the embodiments herein, the target nucleic acid can comprise a rolling circle amplification (RCA) product of a circular or circularized probe that hybridizes to a nucleic acid molecule in the biological sample, wherein the circular or circularized probe can comprise a barcode region.

In any of the embodiments herein, the method can further comprise generating the target nucleic acid. In any of the embodiments herein, the target nucleic acid can be generated in situ in the biological sample.

In any of the embodiments herein, generating the target nucleic acid can comprise: (i) contacting the biological sample with a circular probe or a circularizable probe or probe set that hybridizes to a nucleic acid molecule in the biological sample; and (ii) performing a rolling circle amplification (RCA) using the circular probe or a circularized probe as template, wherein the circularized probe can be generated by circularizing the circularizable probe or probe set. In any of the embodiments herein, the RCA, e.g., for generating the target nucleic acid, can be primed by the nucleic acid molecule or a portion thereof, or by a primer oligonucleotide added to the biological sample, optionally wherein the primer oligonucleotide can hybridize to the nucleic acid molecule. In any of the embodiments herein, the RCA, e.g., for generating the target nucleic acid, can be primed by the nucleic acid molecule or a portion thereof. In any of the embodiments herein, the RCA, e.g., for generating the target nucleic acid, can be primed by a primer oligonucleotide added to the biological sample. In any of the embodiments herein, the primer oligonucleotide can hybridize to the nucleic acid molecule. In any of the embodiments herein, the RCA, e.g., for generating the target nucleic acid, can be performed for greater than about 15 minutes, greater than about 30 minutes, greater than about one hour, greater than about two hours, or greater than about three hours. In any of the embodiments herein, the RCA, e.g., for generating the target nucleic acid, can be performed for greater than about 15 minutes. In any of the embodiments herein, the RCA, e.g., for generating the target nucleic acid, can be performed for greater than about 30 minutes. In any of the embodiments herein, the RCA, e.g., for generating the target nucleic acid, can be performed for greater than about one hour. In any of the embodiments herein, the RCA, e.g., for generating the target nucleic acid, can be performed for greater than about 15 minutes, greater than about 30 minutes, greater than about two hours. In any of the embodiments herein, the RCA, e.g., for generating the target nucleic acid, can be performed for greater than about 15 minutes, greater than about 30 minutes, greater than about three hours.

In any of the embodiments herein, the target nucleic acid can comprise between about 1,000 and about 5,000, between about 5,000 and about 10,000, or more than 10,000 copies of the target sequence. In any of the embodiments herein, the target nucleic acid can comprise between about 1,000 and about 5,000 copies of the target sequence. In any of the embodiments herein, the target nucleic acid can comprise between about 5,000 and about 10,000 copies of the target sequence. In any of the embodiments herein, the target nucleic acid can comprise more than 10,000 copies of the target sequence.

In any of the embodiments herein, the target nucleic acid can be in the form of a nanoball. In any of the embodiments herein, the target nucleic acid can be in the form of a nanoball having a diameter of between about 0.5 μm and about 3 μm, between about 0.8 μm and about 1.5 μm, or between about 1 μm and about 1.3 μm. In any of the embodiments herein, the target nucleic acid can be in the form of a nanoball having a diameter of between about 0.5 μm and about 3 μm. In any of the embodiments herein, the target nucleic acid can be in the form of a nanoball having a diameter of between about 0.8 μm and about 1.5 μm. In any of the embodiments herein, the target nucleic acid can be in the form of a nanoball having a diameter of between about 1 μm and about 1.3 μm.

In any of the embodiments herein, the target nucleic acid can be between about 25 and about 35 kilobases, between about 35 and about 45 kilobases, between about 45 and about 55 kilobases, between about 55 and about 65 kilobases, between about 65 and about 75 kilobases, or more than 75 kilobases in length. In any of the embodiments herein, the target nucleic acid can be between about 25 and about 35 kilobases in length. In any of the embodiments herein, the target nucleic acid can be between about 35 and about 45 kilobases in length. In any of the embodiments herein, the target nucleic acid can be between about 45 and about 55 kilobases in length. In any of the embodiments herein, the target nucleic acid can be between about 55 and about 65 kilobases in length. In any of the embodiments herein, the target nucleic acid can be between about 65 and about 75 kilobases in length. In any of the embodiments herein, the target nucleic acid can be more than 75 kilobases in length.

In any of the embodiments herein, the target sequence can comprise a barcode region.

In any of the embodiments herein, the target nucleic acid can be immobilized in the biological sample. In any of the embodiments herein, the target nucleic acid can be crosslinked to one or more other molecules in the biological sample.

In any of the embodiments herein, the method can comprise imaging the biological sample to detect the detectable probe. In any of the embodiments herein, the biological sample can be imaged using fluorescent microscopy.

In any of the embodiments herein, a sequence of the target nucleic acid can be analyzed, e.g., detected, in situ in the biological sample. In any of the embodiments herein, the sequence to be analyzed can comprise a barcode sequence corresponding to an analyte or a portion thereof or a labeling agent for an analyte or portion thereof in the biological sample. In any of the embodiments herein, the sequence to be analyzed can comprise a barcode sequence corresponding to an analyte or a portion thereof.

In any of the embodiments herein, the biological sample can be a processed or cleared biological sample. In any of the embodiments herein, the biological sample can be a tissue sample. In any of the embodiments herein, the tissue sample can be a tissue slice between about 1 μm and about 50 μm in thickness, optionally wherein the tissue slice can be between about 5 μm and about 35 μm in thickness. In any of the embodiments herein, the tissue slice can be between about 5 μm and about 35 μm in thickness.

In any of the embodiments herein, the tissue sample can be embedded in a hydrogel. In any of the embodiments herein, the target nucleic acid can be crosslinked to the hydrogel, to itself, and/or to one or more molecules in the tissue sample. In any of the embodiments herein, the target nucleic acid can be crosslinked to the hydrogel. In any of the embodiments herein, the target nucleic acid can be crosslinked to itself. In any of the embodiments herein, the target nucleic acid can be crosslinked to one or more molecules in the tissue sample. In any of the embodiments herein, the target nucleic acid can comprise one or more functional groups for attachment to the hydrogel, to itself, and/or to one or more molecules in the tissue sample.

In some embodiments, provided herein is a method for analyzing a biological sample, comprising: (a) contacting the biological sample with a detectable probe, wherein the detectable probe is generated in vitro by: (i) hybridizing a circular oligonucleotide or a circularizable oligonucleotide or oligonucleotide set to a binding oligonucleotide, and (ii) performing rolling circle amplification (RCA) using the circular oligonucleotide or a circularized oligonucleotide as template, wherein the circularized oligonucleotide is generated by circularizing the circularizable oligonucleotide or oligonucleotide set, and wherein the RCA is primed by the binding oligonucleotide or a product thereof, wherein the detectable probe comprises (i) a target-binding region comprising a sequence of the binding oligonucleotide or product thereof and (ii) multiple copies of a unit sequence comprising a detection region configured to hybridize to a detectably labeled oligonucleotide, wherein the circular or circularized oligonucleotide comprises a sequence complementary to the detection region; wherein the biological sample comprises a target nucleic acid comprising multiple copies of a target sequence, and the target-binding region of the detectable probe is configured to hybridize to the target sequence, thereby hybridizing the detectable probe to the target nucleic acid; (b) contacting the biological sample with the detectably labeled oligonucleotide, thereby hybridizing the detectably labeled oligonucleotide to the detectable probe; and (c) detecting a signal associated with the detectably labeled oligonucleotide, thereby detecting the target nucleic acid in the biological sample.

In some embodiments, provided herein is a method for analyzing a biological sample, comprising: (a) providing a detectable probe, wherein the detectable probe is generated in vitro by: (i) hybridizing a circular oligonucleotide or a circularizable oligonucleotide or oligonucleotide set to a binding oligonucleotide, and (ii) performing rolling circle amplification (RCA) using the circular oligonucleotide or a circularized oligonucleotide as template, wherein the circularized oligonucleotide is generated by circularizing the circularizable oligonucleotide or oligonucleotide set, and wherein the RCA is primed by the binding oligonucleotide or a product thereof, wherein the detectable probe comprises (i) a target-binding region comprising a sequence of the binding oligonucleotide or product thereof and (ii) multiple copies of a unit sequence comprising a detection region configured to hybridize to a detectably labeled oligonucleotide, wherein the circular or circularized oligonucleotide comprises a sequence complementary to the detection region; (b) contacting the biological sample with the detectable probe, wherein the biological sample comprises a target nucleic acid comprising multiple copies of a target sequence, and the target-binding region of the detectable probe is configured to hybridize to the target sequence, thereby hybridizing the detectable probe to the target nucleic acid; (c) contacting the biological sample with the detectably labeled oligonucleotide, thereby hybridizing the detectably labeled oligonucleotide to the detectable probe; and (d) detecting a signal associated with the detectably labeled oligonucleotide, thereby detecting the target nucleic acid in the biological sample.

In some embodiments, provided herein is a method for analyzing a biological sample, comprising: (a) generating a detectable probe, wherein the generating comprises: (i) hybridizing a circular oligonucleotide or a circularizable oligonucleotide or oligonucleotide set to a binding oligonucleotide, and (ii) performing rolling circle amplification (RCA) using the circular oligonucleotide or a circularized oligonucleotide as template, wherein the circularized oligonucleotide is generated by circularizing the circularizable oligonucleotide or oligonucleotide set, and wherein the RCA is primed by the binding oligonucleotide or a product thereof, wherein the generated detectable probe comprises (i) a target-binding region comprising a sequence of the binding oligonucleotide or product thereof and (ii) multiple copies of a unit sequence comprising a detection region configured to hybridize to a detectably labeled oligonucleotide, wherein the circular or circularized oligonucleotide comprises a sequence complementary to the detection region; (b) contacting the biological sample with the generated detectable probe, wherein the biological sample comprises a target nucleic acid comprising multiple copies of a target sequence, and the target-binding region of the detectable probe is configured to hybridize to the target sequence, thereby hybridizing the detectable probe to the target nucleic acid; (c) contacting the biological sample with the detectably labeled oligonucleotide, thereby hybridizing the detectably labeled oligonucleotide to the detectable probe; and (d) detecting a signal associated with the detectably labeled oligonucleotide, thereby detecting the target nucleic acid in the biological sample.

In any of the embodiments herein, the target nucleic acid can be in an RCA product. In any of the embodiments herein, the target nucleic acid can be a complex comprising an initiator and an amplifier for hybridization chain reaction (HCR). In any of the embodiments herein, the target nucleic acid can be in a complex comprising an initiator and an amplifier for linear oligonucleotide hybridization chain reaction (LO-HCR). In any of the embodiments herein, the target nucleic acid can be in a primer exchange reaction (PER) product. In any of the embodiments herein, the target nucleic acid can be in a complex comprising a pre-amplifier and an amplifier for branched DNA (bDNA). In any of the embodiments herein, the target nucleic acid can be an intermediate probe that directly or indirectly binds to a primary probe which in turn hybridizes to a cellular nucleic acid molecule. In any of the embodiments herein, the target nucleic acid can be an intermediate probe that directly binds to a primary probe which in turn hybridizes to a cellular nucleic acid molecule.

In some embodiments, provided herein is a detectable probe comprising (i) a target-binding region that hybridizes to a target sequence and (ii) a concatemeric region comprising multiple copies of a unit sequence, wherein the concatemeric region is a rolling circle amplification (RCA) product, and optionally wherein the unit sequence comprises a detection region capable of binding directly or indirectly to a detectably labeled oligonucleotide. In any of the embodiments herein, the unit sequence can comprise a detection region capable of binding directly or indirectly to a detectably labeled oligonucleotide. In any of the embodiments herein, the detection region can be capable of binding directly to the detectably labeled oligonucleotide.

In any of the embodiments herein, the detectable probe can comprise a detectable label. In any of the embodiments herein, the detectable probe can comprise one or more detectably labelled nucleotides, optionally wherein the one or more detectably labelled nucleotides can be incorporated into the concatemeric region during RCA. In any of the embodiments herein, the one or more detectably labelled nucleotides can be incorporated into the concatemeric region during RCA.

In any of the embodiments herein, the unit sequence can comprise a detection region capable of (i) hybridizing to a detectably labeled oligonucleotide or (ii) hybridizing to an intermediate probe which can be capable of hybridizing to a detectably labeled oligonucleotide. In any of the embodiments herein, the unit sequence can comprise a detection region capable of hybridizing to a detectably labeled oligonucleotide. In any of the embodiments herein, the unit sequence can comprise a detection region capable of hybridizing to an intermediate probe which can be capable of hybridizing to a detectably labeled oligonucleotide.

In any of the embodiments herein, the concatemeric region can comprise between about 10 and about 100 or between about 100 and about 1,000 copies of the unit sequence. In any of the embodiments herein, the concatemeric region can comprise between about 10 and about 100 copies of the unit sequence. In any of the embodiments herein, the concatemeric region can comprise between about 100 and about 1,000 copies of the unit sequence.

In any of the embodiments herein, the concatemeric region, optionally the unit sequence, can further comprise the target-binding region. In any of the embodiments herein, the unit sequence can further comprise the target-binding region.

In any of the embodiments herein, the concatemeric region can comprise copies of the detection region and copies of the target-binding region at a ratio of between about 1,000:1 and about 1:1 or between about 100:1 and about 1:1. In any of the embodiments herein, the concatemeric region can comprise copies of the detection region and copies of the target-binding region at a ratio of between about 1,000:1 and about 1:1. In any of the embodiments herein, the concatemeric region can comprise copies of the detection region and copies of the target-binding region at a ratio of between about 100:1 and about 1:1.

In any of the embodiments herein, the concatemeric region, optionally the unit sequence, can further comprise a cleavage site. In any of the embodiments herein, the unit sequence, can further comprise a cleavage site.

In any of the embodiments herein, the concatemeric region can be in the form of a nanoball. In any of the embodiments herein, the concatemeric region can be in the form of a nanoball having a diameter of between about 0.1 μm and about 3 μm, between about 0.1 μm and about 0.5 μm, between about 0.2 μm and about 0.3 μm, or between about 0.3 μm and about 0.4 μm. In any of the embodiments herein, the concatemeric region can be in the form of a nanoball having a diameter of between about 0.1 μm and about 1.5 μm. In any of the embodiments herein, the concatemeric region can be in the form of a nanoball having a diameter of between about 0.15 μm and about 1 μm. In any of the embodiments herein, the concatemeric region can be in the form of a nanoball having a diameter of between about 0.2 μm and about 0.5 μm. In any of the embodiments herein, the concatemeric region can be in the form of a nanoball having a diameter of between about 0.3 μm and about 0.4 μm.

In any of the embodiments herein, the concatemeric region can be between about 1 and about 15 kilobases or between about 15 and about 25 kilobases in length. In any of the embodiments herein, the concatemeric region can be between about 1 and about 15 kilobases in length. In any of the embodiments herein, the concatemeric region can be between about 15 and about 25 kilobases in length.

In some embodiments, provided herein is a kit, comprising: (i) the detectable probe of any of the embodiments herein, and (ii) a detectably labeled oligonucleotide that is capable of binding directly or indirectly to the detection region of the detectable probe. In any of the embodiments herein, the detectably labeled oligonucleotide can be capable of binding directly to the detection region of the detectable probe.

In any of the embodiments herein, the kit can further comprise: (iii) a primary probe capable of binding to an analyte in the biological sample and hybridizing to an intermediate probe, and (iv) the intermediate probe which is capable of hybridizing to the target-binding region of the detectable probe.

### BRIEF DESCRIPTION OF THE DRAWINGS

The drawings illustrate certain features and advantages of this disclosure. These embodiments are not intended to limit the scope of the appended claims in any manner.

FIGS. 1A-1B depict exemplary oligonucleotides for generating a detectable probe that comprises a concatemeric region with multiple copies of a unit sequence. The detectable probe generated using the depicted oligonucleotides comprises detectably labelled nucleotides.

FIG. 1C depicts in situ hybridization of the pre-formed detectable probe to target nucleic acids with multiple copies of a target sequence that hybridizes to the detectable probe. The depicted detectable probe comprises detectably labelled nucleotides.

FIGS. 2A-2G depict exemplary oligonucleotides for generating a detectable probe that comprises a concatemeric region with multiple copies of a unit sequence. The detectable probe generated using the depicted oligonucleotides comprises a detection region.

FIG. 2H depicts in situ hybridization of the pre-formed detectable probe to target nucleic acids. The detectable probe can include multiple copies of one or more detection regions and can be detected with detectably labelled oligonucleotides that hybridize to a sequence of the one or more detection regions.

FIGS. 3A-3B depict in situ hybridization of a plurality of detectable probes in sequential cycles to decode the combinations of barcodes in hybridized primary probes and detect the analytes that the primary probes bind to. The steps depicted FIG. 3B follow the steps depicted in FIG. 3A.

FIG. 4A depicts an example of generating multiple libraries of detectable probes for use in sequential hybridization.

FIGS. 4B-4C depict in situ hybridization of the multiple libraries of detectable probes in sequential cycles to decode the barcodes in the detectable probes and detect the analytes that the barcodes correspond to. The steps depicted FIG. 4C follow the steps depicted in FIG. 4B.

### DETAILED DESCRIPTION

All publications, comprising patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, patent applications, published applications, and other publications that are herein incorporated by reference, the definition set forth herein prevails over the definition that is incorporated herein by reference.

The section headings used herein are for organizational purposes only and are not to be construed as limiting the subject matter described.

### I. Overview

Advances in nucleic acid detection methods, such as single molecule fluorescent hybridization (smFISH), have enabled nanoscale-resolution imaging of analytes such as RNA in cells and tissues. In some cases, however, fluorescent signals can be dim (e.g., in smFISH) while background fluorescence can be high (e.g., in formalin-fixation and paraffin-embedding (FFPE) samples), with large variability depending on factors such as the tissue type, sample age, and fixation conditions. Hence, imaging at high magnification may be required, and only a small area of the sample is usually imaged. Signal amplification can be used to improve signal-to-noise ratio and allow more accurate and efficient analyte detection. Provided herein include detectable probes comprising signal amplification components, e.g., a concatemeric region comprising fluorescent labels (e.g., on nucleotides incorporated into the detectable probes) and/or multiple copies of a unit sequence each capable of hybridizing to one or more intermediate probes and/or fluorescently labeled probes. In some embodiments, the unit sequence comprises a detection region capable of hybridizing to the one or more intermediate probes and/or the fluorescently labeled probes. These detectable probes can be generated and/or pre-assembled with other probes (e.g., fluorescently labeled probes) prior to contacting with target nucleic acids in a sample. For instance, the detectable probes can be generated using a short rolling circle amplification (RCA) reaction in bulk solution (e.g., in a reagent container) and then purified for use in analyte detection in situ, for example, by using the detectable probes in a branched structure for signal amplification and/or to detect an RCA product.

Provided herein in some aspects are methods for analyzing a biological sample. In some embodiments, the biological sample is contacted with a pre-formed detectable probe that comprises a concatemeric region with multiple copies of a unit sequence. In some embodiments, the methods further include steps of providing and/or generating the detectable probe prior to contact with the biological sample. Also provided herein in some aspects are detectable probes that include a concatemeric region with multiple copies of a unit sequence, as well as kits containing the provided detectable probes. In some embodiments, the detectable probe or a portion thereof is generated using RCA, prior to contacting the detectable probe with the biological sample. In some embodiments, the concatemeric region is an amplification product. In some embodiments, the concatemeric region is a rolling circle amplification product (RCP). In some instances, a target nucleic acid comprising one or more target sequence(s) for a detectable probe disclosed herein (e.g., comprising a concatemeric region comprising multiple copies of a unit sequence) is in a hybridization complex that comprises one or more signal amplification components (e.g., primary probe, intermediate probes, etc.).

In some embodiments, the detectable probe comprises a detectable label. For instance, in some embodiments, the detectable probe comprises one or more detectably labelled nucleotides. In some embodiments, the one or more detectably labelled nucleotides are incorporated into the concatemeric region during generation of the concatemeric region, e.g., during RCA. In some embodiments, the unit sequence of the concatemeric region comprises a detection region capable of binding directly or indirectly to a detectably labeled oligonucleotide. In some embodiments, the detection region is capable of binding directly to the detectably labeled oligonucleotide. In some instances, the detection region of the detectable probe comprises a sequence complementary to a sequence of the detectably labeled oligonucleotide. In some embodiments, the concatemeric region comprises multiple copies of the detection region. In some embodiments, the methods further include contacting the biological sample with the detectably labeled oligonucleotide, thereby hybridizing the detectably labeled oligonucleotide to the detectable probe.

In some embodiments, the detectable probe comprises a target-binding region that hybridizes to a target sequence. In some embodiments, the biological sample comprises a target nucleic acid that comprises the target sequence. In some aspects, the concatemeric region of the detectable probe improves the sensitivity with which the target sequence can be detected, for instance detected in situ in the biological sample. In some aspects, the number of detectably labelled nucleotides incorporated into the concatemeric region improves the signal to be detected following hybridization of the detectable probe to the target sequence. Similarly, the number of detection regions incorporated into the concatemeric region can increase the number of detectably labeled oligonucleotides that can be hybridized to the detectable probe, thereby improving the signal to be detected following hybridization. For example, each detectable probe can comprise multiple copies of a unit sequence (e.g., each unit sequence comprising a detection region), which can allow a plurality of detectably labeled oligonucleotides to hybridize to each detectable probe, thereby amplifying the signal associated with each target nucleic acid.

In some embodiments, the biological sample comprises a concatemeric target nucleic acid with multiple copies of a unit sequence. In some embodiments, the unit sequence of the biological sample comprises the target sequence. In some embodiments, the concatemeric target nucleic acid comprises multiple copies of the target sequence. In some embodiments, the concatemeric target nucleic acid is an amplification product. In some embodiments, the concatemeric target nucleic acid is in a hybridization complex (e.g., formed from one or more oligonucleotides forming a branched structure, such as comprising one or more primary probes and/or intermediate probes). In some embodiments, the concatemeric target nucleic acid is an RCP. In some embodiments, the concatemeric target nucleic acid is generated or assembled in situ.

In some embodiments, the concatemeric target nucleic acid is generated using probes or probe sets designed to hybridize to a sequence of interest in an analyte (e.g., a cellular nucleic acid) in the biological sample, for instance in an mRNA molecule. In some embodiments, the probes or probe sets comprise a circular or circularizable probe. In some embodiments, the probes or probe sets include a barcode sequence, e.g., a barcode sequence associated with the analyte or sequence of interest. In some embodiments, the barcode sequence is amplified, for instance by RCA of the probes or probe sets or products thereof. In some embodiments, the barcode sequence is amplified non-enzymatically, for instance using oligonucleotides that assemble into a branched hybridization complex. In some embodiments, the target sequence hybridized by the detectable probes is the barcode sequence. In some instances, the target sequence is part of a primary probe, intermediate probe, or RCP. In some aspects, the sensitivity with which the sequence of interest is detected is improved by the generation of the concatemeric target nucleic acid and the amplification of the barcode sequence. In some aspects, the sensitivity is further improved using detectable probes having concatemeric regions with detectably labelled nucleotides or multiple copies of detection regions for hybridization of detectably labelled oligonucleotides.

### II. Samples, Analytes, and Target Sequences

### A. Samples

A sample disclosed herein can be or be derived from any biological sample. Methods, probes, and kits disclosed herein may be used for analyzing a biological sample, which may be obtained from a subject using any of a variety of techniques including, but not limited to, biopsy, surgery, and laser capture microscopy (LCM), and generally comprises cells and/or other biological material from the subject. A biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO), or a patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be a nucleic acid sample and/or protein sample. The biological sample can be a carbohydrate sample or a lipid sample. The biological sample can be obtained as a tissue sample, such as a tissue section, biopsy, core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., RCPs) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a biological sample can be attached to a substrate. In some embodiments, a substrate can be any support that is insoluble in aqueous liquid and which allows for positioning of biological samples, analytes, features, and/or reagents (e.g., probes) on the support. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (1) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), for which there are established methods. In some embodiments, cell suspensions and other non-tissue samples can be prepared using FFPE. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse or multiple rinses (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, or combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may in some instances be omitted. Alternatively, acetone fixation can be performed in conjunction with permeabilization steps.

In some embodiments, the methods provided herein comprise one or more post-fixing (also referred to as post-fixation) steps. In some embodiments, one or more post-fixing steps are performed after contacting a sample with a polynucleotide disclosed herein, e.g., one or more probes such as a circular probe or a circularizable probe or probe set (e.g., a padlock probe). In some embodiments, one or more post-fixing steps are performed after a hybridization complex comprising a probe and a target is formed in a sample. In some embodiments, one or more post-fixing steps are performed prior to a ligation reaction disclosed herein, such as the ligation to circularize a circularizable probe or probe set.

In some embodiments, one or more post-fixing steps are performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponding to (e.g., uniquely identifying) the analyte. In some embodiments, the labeling agent can comprise a reporter oligonucleotide comprising one or more barcode sequences.

A post-fixing step may be performed using any suitable fixation reagent disclosed herein, for example, 3% (w/v) PFA in diethyl pyrocarbonate (DEPC)-phosphate buffered saline (PBS).

### (iv) Embedding

As an alternative to paraffin embedding described above, a biological sample can be embedded in any of a variety of other embedding materials to provide structural substrate to the sample prior to sectioning and/or other handling steps. In some cases, the embedding material can be removed, e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, for instance devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle, or a compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include Fei-Mao (FM) and rhodamine (RH) dyes or immunohistochemical reagents specific for cell membrane proteins. In some examples, the stain may include but is not limited to, acridine orange, acid fuchsin, Bismarck brown, carmine, coomassie blue, cresyl violet, 4′,6-diamidino-2-phenylindole (DAPI), eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using H&E staining techniques, Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, for instance Wright's stain, Jenner's stain, Can-Grunwald stain, Leishman stain, or Giemsa stain.

In some embodiments, biological samples can be destained. Methods of destaining or discoloring a biological sample generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, for example as described in Chen et al., Science 347(6221):543-548, 2015, the entire contents of which are incorporated herein by reference.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.), and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9×its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides, and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel comprises a hybrid material, e.g., the hydrogel material comprises elements of both synthetic and natural polymers. Examples of suitable hydrogels are described, for example, in U.S. Pat. Nos. 6,391,937, 9,512,422, and 9,889,422, and in U.S. Patent Publication Nos. 2017/0253918, 2018/0052081, and 2010/0055733, the entire contents of each of which are incorporated herein by reference.

In some embodiments, the hydrogel can form the substrate. In some embodiments, the substrate comprises a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH, and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, and dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked.

(viii) Tissue Permeabilization and Treatment

In some embodiments, a biological sample can be permeabilized, for example to facilitate transfer of species (such as probes) into the sample. If a sample is not permeabilized sufficiently, the amount of probes that enter the sample and bind to analytes therein may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™, or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, or mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods that can be used herein include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, Dnase and Rnase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a pair of first and second probes that is specific for (e.g., specifically hybridizes to) each RNA or cDNA analyte is used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by RCA of a circular product generated in a templated ligation reaction).

### B. Analytes

A biological sample may comprise one or a plurality of analytes of interest. Methods for performing multiplexed assays to analyze two or more different analytes in a single biological sample, as well as probes and kits for performing same, are provided.

The methods, probes, and kits disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule, macromolecule, or chemical compound, including a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g., an affinity binding partner) can be developed. Such a specific binding partner may be a nucleic acid probe (for a nucleic acid analyte) and may lead directly to the generation of a RCA template (e.g., a circularizable probe or probe set, such as a padlock probe). Alternatively, the specific binding partner may be coupled to a nucleic acid, which may be detected using an RCA strategy, e.g., in an assay which uses a circular nucleic acid molecule or generates a circularized nucleic acid molecule which can be the RCA template.

Analytes of particular interest may include nucleic acid molecules (e.g., cellular nucleic acids), such as DNA (e.g., genomic DNA, mitochondrial DNA, plastid DNA, viral DNA, etc.) and RNA (e.g., mRNA, microRNA, rRNA, snRNA, viral RNA, etc.), synthetic and/or modified nucleic acid molecules, (e.g., including nucleic acid domains comprising or consisting of synthetic or modified nucleotides such as LNA, PNA, morpholino, etc.), proteinaceous molecules such as peptides, polypeptides, proteins, or prions, or any molecule which comprises a protein or polypeptide component, etc., or fragments thereof, or a lipid or carbohydrate molecule, or any molecule which comprises a lipid or carbohydrate component. The analyte may be a single molecule or a complex that contains two or more molecular subunits, e.g., including but not limited to protein-DNA complexes, which may or may not be covalently bound to one another, and which may be the same or different. Thus, in addition to cells or microorganisms, such a complex analyte may also be a protein complex or protein interaction. Such a complex or interaction may be a homo- or hetero-multimer. Aggregates of molecules, e.g., proteins, may also be target analytes, for example aggregates of the same protein or different proteins. The analyte may also be a complex between proteins or peptides and nucleic acid molecules such as DNA or RNA, e.g., interactions between proteins and nucleic acids, e.g., regulatory factors, such as transcription factors, and DNA or RNA.

In one aspect, provided herein are a plurality of detectable probes capable of binding to a first single-stranded target sequence in a first target nucleic acid and a second single-stranded target sequence in a second target nucleic acid. In some embodiments, the first single-stranded target sequence is not identical to the second single-stranded target sequence. In another aspect, the first single-stranded target sequence is identical to the second single-stranded target sequence. In some embodiments, the second single-stranded target sequence is comprised in the same target nucleic acid (e.g., a nucleic acid analyte or product thereof, a reporter oligonucleotide or product thereof, a probe that directly or indirectly binds to the nucleic acid analyte or product thereof or the reporter oligonucleotide or product thereof, or a product of the probe) as the first single-stranded target sequence. Alternatively, the second single-stranded target sequence is in some embodiments comprised in a different target nucleic acid from the first single-stranded target sequence.

### (i) Endogenous Analytes

In some embodiments, the analyte is endogenous to a biological sample and can include nucleic acid analytes and non-nucleic acid analytes. Methods, probes, and kits disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation, or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods, probes, and kits disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods, probes, and kits for analyzing endogenous analytes (e.g., RNA, ssDNA, and cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence) that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. In some embodiments, an analyte binding moiety barcode is a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods described herein, one or more labeling agents capable of binding to or otherwise coupling to one or more features may be used to characterize analytes, cells, and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Patent Publication No. 2019/0177800; and U.S. Patent Publication No. 2019/0367969, the entire contents of each of which are incorporated herein by reference.

In some embodiments, an analyte binding moiety comprises one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes comprises a single species of analyte (e.g., a single species of polypeptide). In some embodiments in which the plurality of analytes comprises a single species of analyte, the analyte binding moieties of the plurality of analyte labeling agents are the same. In some embodiments in which the plurality of analytes comprises a single species of analyte, the analyte binding moieties of the plurality of analyte labeling agents are different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes comprises multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such as a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linkers, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, the entire contents of which are incorporated herein by reference. Likewise, protein and peptide biotinylation techniques have been developed and are readily available. See, e.g., U.S. Pat. No. 6,265,552, the entire contents of which are incorporated herein by reference. Furthermore, click reaction chemistry may be used to couple reporter oligonucleotides to labeling agents. Commercially available kits, such as those from Thunderlink and Abcam, and any suitable technique may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide can couple the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods, probes, and kits for analyzing one or more products of an endogenous analyte and/or a labeling agent in a biological sample. In some embodiments, an endogenous analyte (e.g., a viral or cellular DNA or RNA) or a product thereof (e.g., a hybridization product, a ligation product, an extension product (e.g., by a DNA or RNA polymerase), a replication product, a transcription/reverse transcription product, and/or an amplification product such as an RCP) is analyzed. In some embodiments, an endogenous analyte, e.g., a target nucleic acid, can be detected directly or indirectly using any of the detectable probes provided herein. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as an RCP) of a labeling agent that directly or indirectly binds to an analyte in the biological sample is analyzed.

In any embodiment described herein, the analyte can comprise or be associated with a target sequence. In some embodiments, the target nucleic acid and the target sequence therein may be endogenous to the sample, generated in the sample, added to the sample, or associated with an analyte in the sample. In some embodiments, the target sequence is a single-stranded target sequence (e.g., a sequence in an RCP). In some embodiments, the target sequence is a single-stranded target sequence (e.g., in a probe bound directly or indirectly to the analyte). In some embodiments, the target sequence is a single-stranded target sequence in a primary probe that binds to an analyte of interest in the biological sample. In some embodiments, the target sequence is a single-stranded target sequence in an intermediate probe which directly or indirectly binds to a primary probe or product thereof, where the primary probe binds to an analyte of interest in the biological sample. In some embodiments, the target sequence is a single-stranded target sequence in a secondary probe that binds to the primary probe or product thereof. In some embodiments, the analytes comprises one or more single-stranded target sequences.

a. Hybridization

In some embodiments, the product of an endogenous analyte and/or a labeling agent is a hybridization product comprising the pairing of substantially complementary or complementary nucleic acid sequences within two different molecules, one of which is the endogenous analyte or the labeling agent (e.g., reporter oligonucleotide attached thereto). The other molecule can be another endogenous molecule or an exogenous molecule such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent, and each probe may comprise one or more barcode sequences. In some instances, various probes and probe sets can be used to generate a product comprising a target sequence that can be hybridized by the detectable probes described herein. In some instances, the probe or probe set is a circularizable probe or probe set comprising a barcode region comprising one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

Specific probe designs can vary depending on the application. For instance, the probe or probe set can comprise a circularizable probe that does not require gap filling to circularize upon hybridization to a template (e.g., a target nucleic acid and/or a probe such as a splint), a gapped circularizable probe (e.g., one that requires gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), a probe or probe set for proximity ligation (such as those described in U.S. Pat. Nos. 7,914,987 and 8,580,504, the entire contents of each of which are incorporated herein by reference, and probes for Proximity Ligation Assay (PLA) for the simultaneous detection and quantification of nucleic acid molecules and protein-protein interactions), or any suitable combination thereof. In some embodiments, the probe or probe set can comprise a probe that is ligated to itself or another probe using DNA-templated and/or RNA-templated ligation. In some embodiments, the probe or probe set can be a DNA molecule and can comprise one or more other types of nucleotides, modified nucleotides, and/or nucleotide analogues, such as one or more ribonucleotides. In some embodiments, the ligation can be a DNA ligation on a DNA template. In some embodiments, the ligation can be a DNA ligation on an RNA template, and the probes can comprise RNA-templated ligation probes. In some embodiments, the probe or probe set can comprise a circularizable probe, such as a padlock-like probe or probe set, such as one described in U.S. Patent Application Nos. 2019/0055594, 2021/0164039, 2016/0108458, or 2020/0224243, the entire contents of each of which are incorporated herein by reference. Any suitable combination of the probe or probe set designs described herein can be used.

In some embodiments, the probe or probe set can comprise two or more parts. In some cases, the probe or probe set can comprise one or more features of and/or be modified based on: a split FISH probe or probe set described in International PCT Patent Publication No. WO 2021/167526A1 or Goh et al., “Highly specific multiplexed RNA imaging in tissues with split-FISH,” Nat Methods 17(7):689-693 (2020), the entire contents of each of which are incorporated herein by reference; a Z-probe or probe set, such as one described in U.S. Pat. Nos. 7,709,198 B2, 8,604,182 B2, 8,951,726 B2, and 8,658,361 B2, or Tripathi et al., “Z Probe, An Efficient Tool for Characterizing Long Non-Coding RNA in FFPE Tissues,” Noncoding RNA 4(3):20 (2018), the entire contents of each of which are incorporated herein by reference; an HCR initiator or amplifier, such as one described in U.S. Pat. No. 7,632,641 B2, U.S. Patent Publication No. 2017/0009278 A1, U.S. Pat. No. 10,450,599 B2, Dirks and Pierce, “Triggered amplification by hybridization chain reaction,” PNAS 101(43):15275-15278 (2004), Chemeris et al., “Real-time hybridization chain reaction,” Dokl. Biochem 419:53-55 (2008), Niu et al., “Fluorescence detection for DNA using hybridization chain reaction with enzyme-amplification,” Chem Commun (Camb) 46(18):3089-91 (2010), Choi et al., “Programmable in situ amplification for multiplexed imaging of mRNA expression,” Nat Biotechnol 28(11):1208-12 (2010), Song et al., “Hybridization chain reaction-based aptameric system for the highly selective and sensitive detection of protein,” Analyst 137(6):1396-401 (2012), Choi et al., “Third-generation in situ hybridization chain reaction: multiplexed, quantitative, sensitive, versatile, robust,” Development 145(12): dev165753 (2018), or Tsuneoka and Funato, “Modified in situ Hybridization Chain Reaction Using Short Hairpin DNAs,” Front Mol Neurosci 13:75 (2020), the entire contents of each of which are incorporated herein by reference; a PLAYR probe or probe set, such as one described in U.S. Patent Publication No. 2016/0108458 A1 or Frei et al., “Highly multiplexed simultaneous detection of RNAs and proteins in single cells,” Nat Methods 13(3):269-75 (2016), the entire contents of each of which are incorporated herein by reference; a PUSH probe or probe set, such as one described in U.S. Patent Publication No. 2020/0224243 A1 or Nagendran et al., “Automated cell-type classification in intact tissues by single-cell molecular profiling,” eLife 7:e30510 (2018), the entire contents of each of which are incorporated herein by reference; a RollFISH probe or probe set such as one described in Wu et al., “RollFISH achieves robust quantification of single-molecule RNA biomarkers in paraffin-embedded tumor tissue samples,” Commun Biol 1, 209 (2018), the entire contents of which are incorporated herein by reference; a MERFISH probe or probe set, such as one described in U.S. Patent Publication No. 2022/0064697 A1 or Chen et al., “Spatially resolved, highly multiplexed RNA profiling in single cells,” Science 348(6233):aaa6090 (2015), the entire contents of each of which are incorporated herein by reference; or a primer exchange reaction (PER) probe or probe set, such as one described in U.S. Patent Publication No. 2019/0106733 A1, the entire contents of which are incorporated herein by reference. In some embodiments, the probe or probe set comprises one or more features and/or is modified to allow for generation and detection of a first signal that does not comprise a nucleic acid amplification step (e.g., the first signal can be an smFISH signal) and generation and detection of a second signal that comprises an amplification step (e.g., extension and/or amplification catalyzed by a polymerase).

b. Ligation

In some embodiments, a product of an endogenous analyte and/or a labeling agent is a ligation product that may comprise a target sequence that can be hybridized by the detectable probes described herein. In some embodiments, the ligation product is formed between two or more endogenous analytes. In some embodiments, the ligation product is formed between an endogenous analyte and a labeling agent. In some embodiments, the ligation product is formed between two or more labeling agent. In some embodiments, the ligation product is an intramolecular ligation of an endogenous analyte. In some embodiments, the ligation product is an intramolecular ligation of a labeling agent or probe, for example, the circularization of a circularizable probe or probe set upon hybridization to a target sequence. The target sequence can be comprised in an endogenous analyte (e.g., nucleic acid such as genomic DNA or mRNA) or a product thereof (e.g., cDNA from a cellular mRNA transcript), or in a labeling agent (e.g., the reporter oligonucleotide) or a product thereof.

In some embodiments, the probe or probe set for generating the ligation product is suitable for DNA-templated ligation, such as cDNA-templated ligation. See, e.g., U.S. Pat. No. 8,551,710, the entire contents of which are incorporated herein by reference. In some embodiments, the probe or probe set for generating the ligation product is suitable for RNA-templated ligation. See, e.g., U.S. Patent Publication No. 2020/0224244, the entire contents of which are incorporated herein by reference. In some embodiments, the probe set is a SNAIL probe set. See, e.g., U.S. Patent Publication No. 2019/0055594, the entire contents of which are incorporated herein by reference.

In some embodiments, the probe or probe set for generating the ligation product is suitable for a multiplexed proximity ligation assay. See, e.g., U.S. Patent Publication No. 2014/0194311, the entire contents of which are incorporated herein by reference. In some embodiments, the probe or probe set for generating the ligation product is suitable for proximity ligation, for instance a proximity ligation assay for RNA (e.g., PLAYR) probe set. See, e.g., U.S. Patent Publication No. 2016/0108458, the entire contents of which are incorporated herein by reference. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Patent Publication No. 2020/0224243, the entire contents of which are incorporated herein by reference.

In some embodiments, a circular or circularizable probe or probe set may be used to analyze a reporter oligonucleotide, which may generated using proximity ligation or be subjected to proximity ligation. In some examples, the reporter oligonucleotide of a labeling agent that specifically recognizes a protein can be analyzed using in situ hybridization (e.g., sequential hybridization) and/or in situ sequencing (e.g., using circular or circularizable probes and rolling circle amplification of circular or circularized probes). Further, the reporter oligonucleotide of the labeling agent and/or a complement thereof and/or a product (e.g., a hybridization product, a ligation product, an extension product (e.g., by a DNA or RNA polymerase), a replication product, a transcription/reverse transcription product, and/or an amplification product) thereof can be recognized by another labeling agent and analyzed.

In some embodiments, an analyte (a nucleic acid analyte or non-nucleic acid analyte) can be specifically bound by two labeling agents (e.g., antibodies) each of which is attached to a reporter oligonucleotide (e.g., DNA) that can participate in ligation, replication, and sequence decoding reactions, e.g., using a probe or probe set (e.g., a circular probe or a circularizable probe or probe set, such as a padlock probe, a SNAIL probe set, a gapped padlock probe, or a gapped padlock probe and a connector). In some embodiments, the probe set may comprise two or more probe oligonucleotides, each comprising a region that is complementary to each other. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods. (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Publication No. 2002/0051986, the entire contents of which are incorporated herein by reference. In some embodiments, two analytes in proximity can be specifically bound by two labeling agents (e.g., antibodies) each of which is attached to a reporter oligonucleotide (e.g., DNA) that can participate, when in proximity when bound to their respective targets, in ligation, replication, and/or sequence decoding reactions

In some embodiments, one or more reporter oligonucleotides (and optionally one or more other nucleic acid molecules such as a connector) aid in the ligation of the probe. Upon ligation, the probe may form a circularized probe. In some embodiments, one or more suitable probes can be used and ligated, wherein the one or more probes comprise a sequence that is complementary to the one or more reporter oligonucleotides (or portion thereof). The probe may comprise one or more barcode sequences. In some embodiments, the one or more reporter oligonucleotides may serve as a primer for RCA of the circularized probe. In some embodiments, a nucleic acid other than the one or more reporter oligonucleotides is used as a primer for RCA of the circularized probe. For example, a nucleic acid capable of hybridizing to the circularized probe at a sequence other than sequence(s) hybridizing to the one or more reporter oligonucleotide can be used as the primer for RCA. In other examples, the primer in a SNAIL probe set is used as the primer for RCA.

In some embodiments, one or more analytes can be specifically bound by two primary antibodies, each of which is in turn recognized by a secondary antibody each attached to a reporter oligonucleotide (e.g., DNA). Each nucleic acid molecule can aid in the ligation of the probe to form a circularized probe. In some instances, the probe can comprise one or more barcode sequences. Further, the reporter oligonucleotide may serve as a primer for RCA of the circularized probe. The nucleic acid molecules, circularized probes, and RCP can be analyzed using any suitable method disclosed herein for in situ analysis.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases, and single-strand polynucleotide ligases, for example any of the ligases described in EC 6.5.1.1 (ATP-dependent ligases), EC 6.5.1.2 (NAD+-dependent ligases), and EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies), and phage ligases such as T3 DNA ligase, T4 DNA ligase, and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation is a direct ligation. In some embodiments, the ligation is an indirect ligation. “Direct ligation” can mean that the ends of the polynucleotides hybridize immediately adjacently to one another to form a substrate for a ligase enzyme resulting in their ligation to each other (intramolecular ligation). Alternatively, “indirect” can mean that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, a circularizable probe (e.g., a padlock probe), or target nucleic acid. The gap may be a gap of 1 to 60 nucleotides, a gap of 1 to 40 nucleotides, or a gap of 3 to 40 nucleotides. In specific embodiments, the gap may be a gap of about 1, 2, 3, 4, 5, 6, 7, 8, 9 or 10 or more nucleotides, or any range of integers of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases can be active at elevated temperatures, allowing further discrimination by incubating the ligation at a temperature near the melting temperature (Tₘ) of the DNA strands. This can selectively reduce the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations can involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

c. Primer Extension

In some embodiments, a product is a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents). Any of such products of extension may comprise a target sequence that can be hybridized by the detectable probes described herein.

A primer is generally a single-stranded nucleic acid sequence having a 3′ end that can be used as a substrate for a nucleic acid polymerase in a nucleic acid extension reaction. RNA primers are formed of RNA nucleotides, and can be used in RNA synthesis, while DNA primers are formed of DNA nucleotides and can be used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer may, in some cases, refer to a primer binding sequence. In some aspects, a primer extension reaction is a method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labeling agent is an amplification product of one or more polynucleotides, for instance, a circular probe or circularizable probe or probe set. In some embodiments, the amplifying is achieved by performing RCA. In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the RCA is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for RCA include a linear RCA, a branched RCA, a dendritic RCA, or any combination thereof. See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; and U.S. Pat. Nos. 6,054,274, 6,291,187, 6,323,009, 6,344,329, and 6,368,801, the entire contents of each of which are incorporated herein by reference. Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Examples of the modified nucleotides comprise amine-modified nucleotides. In some aspects of the methods, the modified nucleotides are incorporated for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, U.S. Patent Publication Nos. 2016/0024555, 2018/0251833, and 2017/0219465, the contents of each of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers, or chemical groups to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix, preserving their spatial information within the cell and thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to an in situ synthesized hydrogel for tissue clearing, enzyme diffusion, and multiple-cycle sequencing. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, the detection of numerous different analytes may use a RCA-based detection system, e.g., where the signal is provided by generating a RCP from a circular RCA template which is provided or generated in the assay, and the RCP is detected to detect the corresponding analyte. The RCP may thus be regarded as a reporter which is detected to detect the target analyte. However, the RCA template may also be regarded as a reporter for the target analyte; in some aspects, the RCP is generated based on the RCA template and comprises complementary copies of the RCA template. The RCA template can determine the signal which is detected, and can thus be indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g., circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

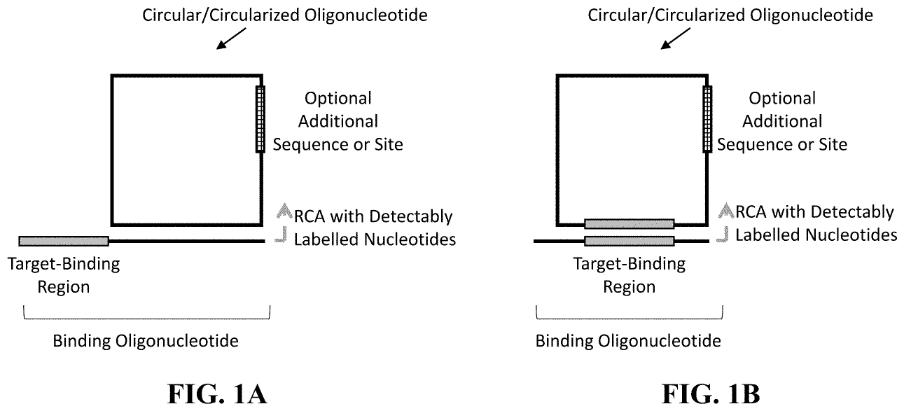

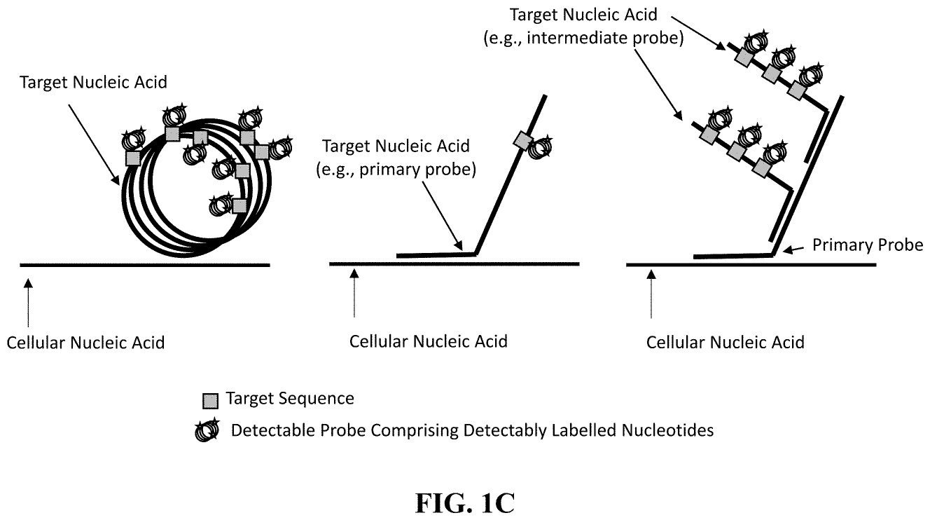

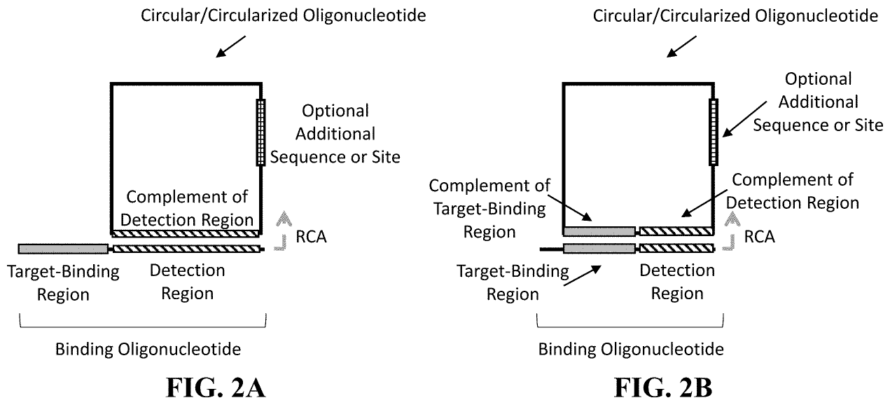

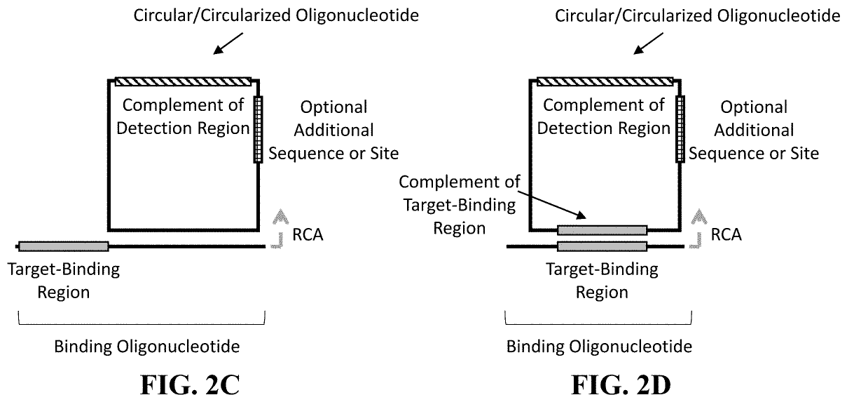

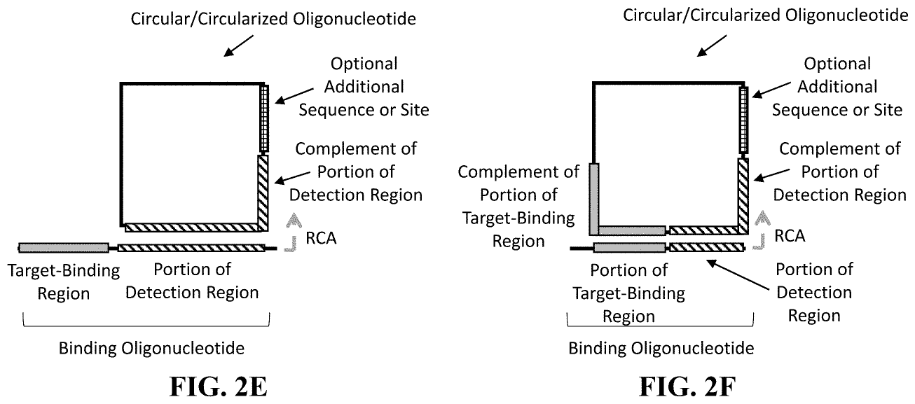

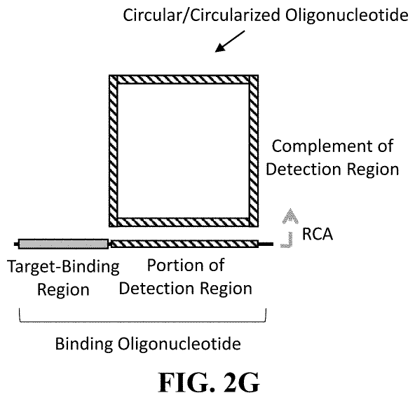

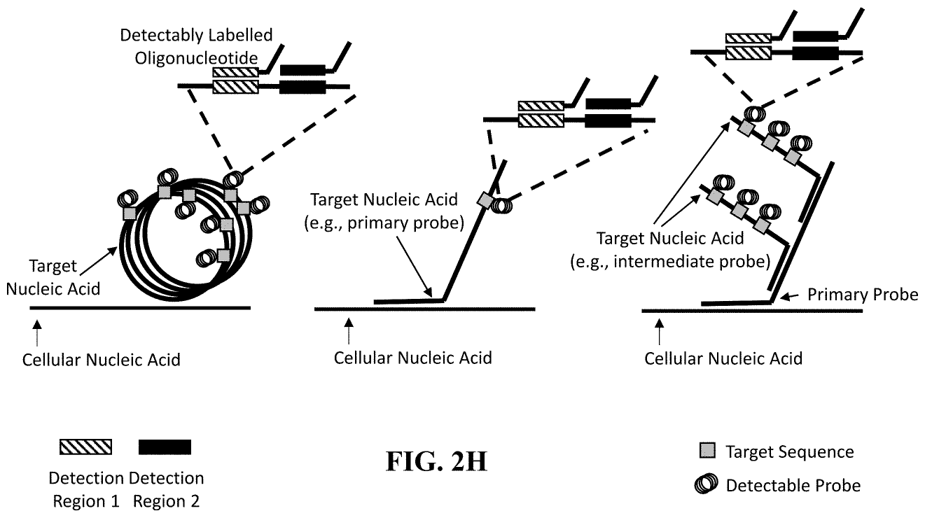

In some embodiments, the product comprises a molecule or a complex generated in a series of reactions, e.g., hybridization, ligation, extension, replication, transcription/reverse transcription, and/or amplification (e.g., RCA), in any suitable combination. For example, a product comprising a target sequence for a detectable probe disclosed herein (e.g., comprising a concatemeric region comprising multiple copies of a unit sequence) may be a hybridization complex formed of a cellular nucleic acid in a sample and an exogenously added nucleic acid probe or a product generated therefrom. The exogenously added nucleic acid probe (e.g., primary probe) may comprise an overhang that does not hybridize to the cellular nucleic acid but hybridizes to another probe (e.g., an intermediate probe) or detectable probes such as depicted in FIGS. 1C and 2H. In other examples, a product comprising a target sequence for a detectable probe disclosed herein (e.g., comprising a concatemeric region comprising multiple copies of a unit sequence) may an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a detectable probe disclosed herein (e.g., comprising a concatemeric region comprising multiple copies of a unit sequence) may be generated using a probe hybridizing to a cellular nucleic acid.

In some instances, a target nucleic acid comprising a target sequence for a detectable probe disclosed herein (e.g., comprising a concatemeric region comprising multiple copies of a unit sequence) is a product that comprises one or more signal amplification components. In some instances, the amplification comprises one or more probe hybridizations and generation of amplified signals associated with the probes (e.g., primary and/or intermediate probes). In some instances, the target nucleic acid (e.g., primary probe as depicted in the middle panels of FIGS. 1C and 2H) comprises one or more target sequence(s) for detectable probe hybridization, such that the signal is amplified by the presence of multiple detectable probes hybridized to the target nucleic acid. In some instances, the target nucleic acid (e.g., intermediate probe as depicted in the right panels of FIGS. 1C and 2H) comprises multiple target sequences for detectable probe hybridization, such that the signal corresponding to a barcode sequence is amplified by the presence of multiple detectable probes hybridized to the target nucleic acid. Exemplary signal amplification methods include targeted assembly of branched structures (e.g., bDNA). In some instances, detection of nucleic acids sequences in situ includes combination of the sequential decoding methods described herein with an assembly for branched signal amplification using the detectable probes provided herein. In some instances, the assembly complex comprises an amplifier hybridized directly or indirectly (via one or more oligonucleotides) to a sequence of a cellular nucleic acid.

### III. Analysis Using Detectable Probes

Provided herein in some embodiments are methods for analyzing a biological sample comprising a target nucleic acid by contacting the biological sample with a pre-formed detectable probe comprising a concatemeric region with multiple copies of a unit sequence. In some embodiments, the methods further include steps of generating the detectable probe prior to contacting with the biological sample. In some embodiments, the methods further includes providing a generated detectable probe prior to contacting with the biological sample. Also provided herein in some aspects are methods using a plurality of detectable probes that each include a concatemeric region with multiple copies of a unit sequence, and detecting and analyzing signals associated with the detectable probes.

### A. Target Sequences

In some instances, a method disclosed herein may comprise detecting signals associated with a target nucleic acid using detectable probes. In some instances, the present disclosure relates to the detection of nucleic acid sequences in situ using probe hybridization and generation of amplified signals associated with the detectable probes. In some instances, the target nucleic acid comprises multiple target sequences for hybridization of a plurality of detectable probes that each comprise a concatemeric region with multiple copies of a unit sequence, such that the signal corresponding to a target nucleic acid is amplified by the presence of multiple detectable probes hybridized to the target nucleic acid. In one example, the target nucleic acid can be an amplification product (e.g., an RCP) comprising multiple copies of a target sequence (e.g., a barcode sequence of the RCP). In one example, the target nucleic acid comprising a target sequence can be in an hybridization complex (e.g., primary probe bound to a cellular nucleic acid). In some instances, the target nucleic acid may comprise one or more copies of the target sequence (e.g., a primary probe bound to a cellular nucleic acid or an intermediate probe bound thereto). In some instances, one or more target nucleic acids are part of an amplification product comprising an assembly of branched nucleic acid structures into a hybridization complex. In some instances, the assembled hybridization complex comprises an amplifier hybridized directly or indirectly (via one or more oligonucleotides) to a cellular nucleic acid analyte. In some instances, the assembly comprises one or more amplifiers each including an amplifier repeating sequence. In some embodiments, the target nucleic acid is any of the nucleic acid analytes described in Section II.B. In some embodiments, the provided methods comprise steps for generating the target nucleic acid, for instance as described in Section II.B for any of the nucleic acid analytes described therein.

In some embodiments, the target nucleic acid comprises multiple copies of the target sequence. In some embodiments, the target nucleic acid is a concatemeric target nucleic acid with multiple copies of the target sequence. In some embodiments, the target nucleic acid is in a hybridization complex with multiple copies of the target sequence. In some embodiments, the target nucleic acid comprises between about 5 and about 50, between about 10 and about 100, between about 100 and about 1,000, between about 1,000 and about 5,000, between about 5,000 and about 10,000, or more than 10,000 copies of the target sequence. In some embodiments, the target nucleic acid comprises between about 1,000 and about 5,000, between about 5,000 and about 10,000, or more than 10,000 copies of the target sequence. In some embodiments, the target nucleic acid is in a hybridization complex that comprises between about 5 and about 50, between about 10 and about 100, between about 100 and about 1,000, between about 1,000 and about 5,000, between about 5,000 and about 10,000, or more than 10,000 copies of the target sequence. In some embodiments, the target nucleic acid comprises between about 1,000 and about 5,000, between about 5,000 and about 10,000, or more than 10,000 copies of the target sequence.

In some embodiments, the target nucleic acid is in the form of a nanoball, e.g., as generated using RCA. In some embodiments, the nanoball has a diameter of between about 0.1 μm and about 3 μm, e.g., between about 0.1 μm and about 0.5 μm (e.g., between about 0.2 μm and about 0.3 μm, or between about 0.3 μm and about 0.4 μm), between about 0.5 μm and about 1 μm, between about 0.8 μm and about 1.3 μm, or between about 1 μm and about 1.5 μm. In some embodiments, the nanoball has a diameter of between about 0.5 μm and about 1 μm, between about 0.8 μm and about 1.3 μm, or between about 1 μm and about 1.5 μm.

In some embodiments, the target nucleic acid is between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length, e.g., between about 45 and about 70 kilobases in length. In some embodiments, the target nucleic acid is between about 25 and about 35 kilobases, between about 35 and about 45 kilobases, between about 45 and about 55 kilobases, between about 55 and about 65 kilobases, between about 65 and about 75 kilobases, or more than 75 kilobases in length, e.g., between about 45 and about 70 kilobases in length.

In some embodiments, the target nucleic acid is an amplification product, for instance any of the amplification products described herein. In some embodiments, the target nucleic acid comprises an RCP, e.g., an RCP generated according to any of the described methods, for instance an RCP of a circular or circularized probe that hybridizes to a nucleic acid molecule in the biological sample. In some embodiments, the target sequence is a barcode sequence or complement thereof of the RCP. In some embodiments, the RCA for generating the RCP of the target nucleic acid is performed for greater than about 15 minutes, greater than about 30 minutes, greater than about one hour, greater than about two hours, or greater than about three hours.

In some aspects, one or more of the target sequences comprises one or more barcode(s), e.g., at least two, three, four, five, six, seven, eight, nine, ten, or more barcodes. Barcodes can spatially-resolve molecular components found in biological samples, for example, within a cell or a tissue sample. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode comprises two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcodes or complements thereof (e.g., barcode sequences or complements thereof comprised by the RCA products herein) can be detected by sequential hybridization and detection with a plurality of detectable probes described herein. In some embodiments, barcode sequences are detected for identification of other molecules including nucleic acid molecules (DNA or RNA) longer than the barcode sequences themselves, as opposed to direct sequencing of the longer nucleic acid molecules. In some embodiments, an N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or products thereof (e.g., RCPs) are detected, rather than endogenous sequences, which can be an efficient read-out in terms of information per cycle of sequencing. Because the barcode sequences are pre-determined, they can also be designed to feature error detection and correction mechanisms, see, e.g., U.S. Patent Publication Nos. 2019/0055594 and 2021/0164039, the entire contents of each of which are incorporated herein by reference.

In some aspects, the provided methods involve analyzing, e.g., detecting or determining, one or more sequences present in a probe and/or in an amplification product, such as in an amplification product of a primary probe (e.g., a linear primary probe, a probe in a hybridization complex, or a circular/circularized probe), which comprises or is associated with one or more barcode sequences. In some embodiments, the analysis comprises detecting a sequence present in the amplification product. In some embodiments, the analysis can be used to correlate a sequence detected in an amplification product to a circular or circularizable probe or probe set (e.g., via a barcode). In some embodiments, the detection of a sequence in an amplification product can provide information regarding the location and/or identity of an associated target nucleic acid (e.g., hybridized by the primary probe) in a sample. In some embodiments, due to amplification of one or more polynucleotides (e.g., a circular or circularizable probe or probe set), particular sequences present in the amplification product or complementary sequences thereof can be detected even when a polynucleotide is present at low levels before the amplification. For example, the number of copies of the barcode sequence(s) and/or a complementary sequence thereof can be increased by virtue of the amplification of a probe comprising the barcode sequence(s) and/or complementary sequence thereof, thereby enabling specific and sensitive detection of a signal indicative of the identity of a short region (e.g., a single nucleotide) of interest in a target nucleic acid. In particular embodiments, the amplification product is an in situ RCP of a circular probe which can be circularized from a circularizable probe or probe set.

In some embodiments, provided herein are methods for assessing one or more target nucleic acids, such as a plurality of different mRNAs, in a biological sample, such as a cell or a tissue sample (such as a tissue section). In some embodiments, the target nucleic acid comprises DNA. In some embodiments, the target nucleic acid comprises RNA. In some embodiments, the circular or circularizable probe or probe set comprises DNA. In some embodiments, the target nucleic acid is RNA, and the circular or circularizable probe or probe set comprises DNA. In some embodiments, the target nucleic acid is RNA, and the circular or circularizable probe or probe set comprises one or more ribonucleotides. In some embodiments, the circular or circularizable probe or probe set comprises primarily deoxyribonucleotide residues and one, two, three, four, or more ribonucleotide residues. In some embodiments, the circular or circularizable probe or probe set comprises no more than two, no more than three, no more than four consecutive ribonucleotide residues. In some embodiments, the one or more ribonucleotide residues are at a 3′ end and/or a 5′ end of the circularizable probe or probe set. In some embodiments, the circularizable probe or probe set comprises a 3′ terminal ribonucleotide residue.

In some aspects, the provided methods are employed for in situ analysis of target nucleic acids, for example for in situ sequencing or multiplexed analysis in intact tissues or a sample with preserved cellular or tissue structure. In some aspects, the provided methods can be used to detect or determine the identity or amount in situ of single nucleotides of interest in target nucleic acids, for instance of single nucleotide polymorphisms of genes of interest.

In some aspects, the provided methods involve a step of contacting, or hybridizing, one or more polynucleotides to a cell or a sample containing a target analyte (e.g., cellular nucleic acid) in order to form a hybridization complex. In some aspects, the provided methods comprise one or more steps of ligating the polynucleotides, for instance of ligating the ends of the circularizable probe, e.g., padlock probe, to form a circularized probe. In some aspects, the provided methods involve a step of amplifying one of the polynucleotides (e.g., a linear probe or a circular/circularized probe produced therefrom), to generate an amplification product. In some instances, the target nucleic acid is an amplification product generated enzymatically. In some instances, the target nucleic acid is an amplification product generated non-enzymatically. In some aspects, the provided methods involve a step of detecting and/or determining the sequence of all or a portion of the amplification product (for example, of one or more barcodes contained in the amplification product) and/or one or more of the polynucleotides with or without amplification, for instance any barcodes contained therein. In some aspects, the provided methods involve performing one or more of the steps described herein, simultaneously and/or sequentially.

In some embodiments, a primary probe (e.g., linear or circular/circularizable probe) disclosed herein comprises 1, 2, 3, 4, 5, 6, 7, 8, 9, 10, 11, 12, 13, 14, 15 or more, 20 or more, 32 or more, 40 or more, or 50 or more barcode sequences. The barcode sequences may be positioned anywhere within the nucleic acid probe. If more than one barcode sequence is present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, or at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases. In some embodiments, the bases are consecutive bases.

In some embodiments, the number of distinct barcode sequences in a population of nucleic acid probes is less than the number of distinct targets (e.g., nucleic acid analytes and/or protein analytes) of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes. In some embodiments, the barcode sequences or any subset thereof in the population of nucleic acid probes can be independently and/or combinatorially detected and/or decoded.

In some embodiments, the methods of the present disclosure include the step of performing RCA to generate a target nucleic acid associated with an analyte (e.g., cellular nucleic acid) of interest. In some embodiments, the method comprises using a circular or circularizable construct hybridized to the analyte of interest to generate the target nucleic acid. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. Any suitable methods and conditions for hybridization of probes, ligation, amplification, and detection may be used, e.g., any as described herein. In any of the embodiments herein, the product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In some embodiments, the circular construct used as template for RCA is formed using ligation. In some embodiments, the circular construct is formed using template primer extension followed by ligation. In some embodiments, the circular construct is formed by providing an insert between ends to be ligated. In some embodiments, the circular construct is formed using a combination of any of the foregoing. In some embodiments, the ligation is a DNA-DNA templated ligation. In some embodiments, the ligation is an RNA-RNA templated ligation. In some embodiments, the ligation is an RNA-DNA templated ligation. In some embodiments, a splint is provided as a template for ligation.

In some embodiments, the circular construct is directly hybridized to the target analyte (e.g., cellular nucleic acid). In some embodiments, the circular construct is formed from a circularizable probe or probe set, such as a padlock probe. In some embodiments, the circular construct is formed from a probe or probe set capable of DNA-templated ligation. See, e.g., U.S. Pat. No. 8,551,710, the entire contents of which are incorporated herein by reference. In some embodiments, the circular construct is formed from a probe or probe set capable of RNA-templated ligation. Exemplary RNA-templated ligation probes and methods are described in U.S. Patent Publication Nos. 2018/0208967 and 2020/0224244, the entire contents of each of which are incorporated herein by reference. In some embodiments, the circular construct is formed from a specific amplification of nucleic acids via intramolecular ligation (e.g., SNAIL) probe set. See, e.g., U.S. Patent Publication No. 2019/0055594, the entire contents of which are incorporated herein by reference. In some embodiments, the circular construct is formed from a probe capable of proximity ligation, for instance a proximity ligation assay for RNA (e.g., PLAYR) probe set. See, e.g., U.S. Patent Publication No. 2016/0108458, the entire contents of which are incorporated herein by reference.

In some embodiments, the circular construct is indirectly hybridized to the target analyte (e.g., cellular nucleic acid). In some embodiments, the circular construct is formed from a probe set capable of proximity ligation, for instance a proximity ligation in situ hybridization (PLISH) probe set. See, e.g., U.S. Patent Publication No. 2020/0224243, the entire contents of which are incorporated herein by reference.

The nature of the ligation reaction depends on the structural components of the polynucleotides used to form the circular or circularizable probe or probe set. In one embodiment, the polynucleotides comprise complementary docking regions that self-assemble the two or more polynucleotides into a circularizable probe that is either ready for ligation because no gaps exist between the docking regions, or is ready for a fill-in process, which will then permit the ligation of the polynucleotides to form the circularizable probe. In another embodiment, the docking regions are complementary to a splint primer. In one embodiment, the splint primer is complementary to one pair of docking regions of two polynucleotides. In another embodiment, the splint primer is complementary to two pairs of docking regions. In one aspect of this embodiment, the splint primer has two regions of complementarity to the docking regions of the polynucleotides that form the circularizable probe. Typically, a splint probe of this embodiment will comprise a first docking region complementary sequence, a spacer, and a second docking region complementary sequence.

In some embodiments, a 3′ end and a 5′ end of the circularizable probe or probe set can be ligated using the target analyte (e.g., cellular nucleic acid such as RNA) as a template. In some embodiments, the 3′ end and the 5′ end are ligated without gap filling prior to ligation. In some embodiments, the ligation of the 3′ end and the 5′ end is preceded by gap filling. The gap may be 1, 2, 3, 4, 5, or more nucleotides.

In some embodiments, a probe disclosed herein (e.g., a circularizable probe, such as a padlock probe) can comprise a 5′ flap which may be recognized by a structure-specific cleavage enzyme, e.g., an enzyme capable of recognizing the junction between single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to the target analyte (e.g., cellular nucleic acid), as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiment, cleavage of the additional sequence 5′ to the first target-specific binding site is performed by a structure-specific cleavage enzyme, e.g., a Flap endonuclease. Suitable Flap endonucleases are described in, for example, Ma et al. 2000. JBC 275, 24693-24700 and in U.S. Patent Publication No. 2020/0224244, the entire contents of each of which are incorporated herein by reference, and may include P. furiosus (Pfu), A. fulgidus (Afu), M jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. In some aspects, a 5′ nuclease enzyme is capable of recognizing a free 5′ end of a single-stranded oligonucleotide and degrading said single-stranded oligonucleotide. In some aspects, 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g., dinucleotides or trinucleotides), or cleaving the entire 5′ single-stranded region, e.g., as described in Lyamichev et al. 1999. PNAS 96, 6143-6148, the entire contents of which are incorporated herein by reference, for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

In any of the embodiments herein, the method can further comprise ligating the ends of the circularizable probe or probe set hybridized to the target analyte (e.g., a cellular nucleic acid such as RNA) to form a circularized probe. In any of the embodiments herein, the method can further comprise generating an RCP of the circularized probe. In any of the embodiments herein, the method can further comprise detecting a signal associated with the RCP in the biological sample using a detectable probe comprising a concatemeric region comprising multiple copies of a unit sequence described herein.

In some embodiments, the circularizing step may comprise ligation selected from the group consisting of enzymatic ligation, chemical ligation, template dependent ligation, and/or template independent ligation. In any of the embodiments herein, the ligation can comprise using a ligase having an RNA-templated DNA ligase activity and/or an RNA-templated RNA ligase activity. In any of the embodiments herein, the ligation can comprise using a ligase selected from the group consisting of a Chlorella virus DNA ligase (PBCV DNA ligase), a T4 RNA ligase, a T4 DNA ligase, and a single-stranded DNA (ssDNA) ligase. In any of the embodiments herein, the ligation can comprise using a PBCV-1 DNA ligase or variant or derivative thereof and/or a T4 RNA ligase 2 (T4 Rnl2) or variant or derivative thereof.

Following formation of, e.g., the circularized probe or otherwise providing a circular probe, in some instances, an amplification primer is added. In other instances, the amplification primer is added with the circular or circularizable probe or probe set. In some instances, the amplification primer may also be complementary to the target nucleic acid and the circular or circularizable probe or probe set (e.g., a SNAIL probe). In some embodiments, a washing step is performed to remove any unbound probes, primers, etc. In some embodiments, the wash is a stringency wash. Washing steps can be performed at any point during the process to remove non-specifically bound probes, probes that have ligated, etc.

Upon addition of a DNA polymerase in the presence of appropriate dNTP precursors and other cofactors, the amplification primer is elongated by replication of multiple copies of the template (e.g., a concatemer of the template is generated). This amplification product can be detected using, e.g., detectable probes comprising a concatemeric region comprising multiple copies of a unit sequence and detection oligonucleotides (e.g., detectably labeled probes) described herein. In any of the embodiments herein, a sequence in the amplification product can be determined or otherwise detected, for example by using detectably labeled probes and imaging. The detection of the amplification products can comprise sequencing by hybridization (e.g., sequential fluorescent in situ hybridization). In some instances, detection using, e.g., detectable probes comprising a concatemeric region comprising multiple copies of a unit sequence described herein.

In any of the embodiments herein, the product can be immobilized in the biological sample. In any of the embodiments herein, the product can be crosslinked to one or more other molecules (e.g., a cellular molecule or an extracellular molecule) in the biological sample. Any suitable methods for tethering and immobilization may be used, e.g., any described in Section II.

### B. Detectable Probes

Provided herein in some embodiments is a detectable probe. In some embodiments, the provided methods involve contacting the biological sample with the detectable probe. In some embodiments, the detectable probe contains a detectable label. In some embodiments, the detectable probe can be used to detect a target nucleic acid, e.g., a cellular nucleic acid, one or more probe(s), and/or amplification products (e.g., amplicon) described herein. In some embodiments, the provided methods include detecting a signal associated with the detectable probe, thereby detecting a target nucleic acid or a sequence thereof in the biological sample. In some embodiments, the methods involve incubating the detectable probe with the sample, washing unbound detectable probe, and detecting the detectable probe, e.g., by imaging. In some embodiments, a detectable probe may bind directly or indirectly to a cellular nucleic acid, e.g., bind to a probe (e.g., a primary or intermediate probe) and/or amplification products.

In some embodiments, the detectable probe or a portion thereof is generated using RCA. In some embodiments, the concatemeric region of the detectable probe is an amplification product. In some embodiments, the concatemeric region is an RCP.

The probes may comprise any of a variety of entities that can hybridize to a nucleic acid, typically by Watson-Crick base pairing, such as DNA, RNA, LNA, PNA, etc., depending on the application. In some embodiments, the detectable probe comprises a target-binding region that hybridizes to a target sequence of a target nucleic acid. The detectable probe may be able to bind to a specific target nucleic acid. In some embodiments, the detectable probes may comprise a detectable label, and/or can be detected by using detectably labeled oligonucleotides that bind to the detectable probes.

In any of the embodiments herein, the detecting step can comprise contacting the biological sample with one or more intermediate probes that directly or indirectly hybridize to the target nucleic acid, e.g., an amplification product (e.g., RCP or hybridization complex), wherein the one or more intermediate probes are detectable using one or more detectable probes. In any of the embodiments herein, the detecting step can further comprise dehybridizing the one or more intermediate probes and/or the one or more detectable probes from the target nucleic acid, e.g., an amplification product (e.g., RCP or hybridization complex). In any of the embodiments herein, the contacting and dehybridizing steps can be repeated with the one or more intermediate probes, the one or more detectable probes, one or more other intermediate probes, and/or one or more other detectable probes. In some cases, the repeated contacting, detection and dehybridizing steps allows detection of barcode sequences or complements thereof and identification of the corresponding sequences of signal codes (e.g., fluorophore sequences assigned to the corresponding barcode sequences or complements thereof).

In any of the embodiments herein, the detecting step can comprise contacting the biological sample with one or more detectable probes that directly or indirectly hybridize to the target nucleic acid, e.g., an amplification product (e.g., RCP or hybridization complex), and dehybridizing the one or more detectable probes from the target nucleic acid, e.g., an amplification product (e.g., RCP or hybridization complex). In any of the embodiments herein, the contacting and dehybridizing steps can be repeated with the one or more detectable probes and/or one or more other detectable probes that directly or indirectly hybridize to the target nucleic acid.

In some embodiments, the detectable probe is a pre-formed probe that is generated prior to contacting the biological sample. In some embodiments, the detectable probe is generated ex situ, e.g., outside the biological sample, for instance, in bulk solution in a container. In any of the embodiments herein, the detectable probe can be produced in vitro. In some embodiments, the detectable probe is formed in a separate mixture from the biological sample.

In some embodiments, the detectable probe comprises a target-binding region that hybridizes to a target sequence (e.g., described in Section III.A). In some embodiments, the target sequence is contained in a target nucleic acid within the biological sample. The target nucleic acid can be associated with any of the analytes described herein. In some embodiments, the target nucleic acid is associated with a cellular nucleic acid molecule, e.g., genomic DNA, mRNA, or cDNA. In some embodiments, the target nucleic acid is associated with an mRNA molecule. In some embodiments, the target nucleic acid is associated with a reporting oligonucleotide of a labeling agent, e.g., any of the labeling agents described herein. In some embodiments, the target nucleic acid is an exogenous molecule added to the sample or is a molecule that has been generated in situ in the sample.

In some embodiments, the detectable probe comprises a concatemeric region with multiple copies of a unit sequence. In some embodiments, the concatemeric region comprises between about 10 and about 100, between about 50 and about 500, between about 100 and about 1,000, between about 1,000 and about 5,000, between about 5,000 and about 10,000, or more than 10,000 copies of the unit sequence. In some embodiments, the concatemeric region comprises between about 10 and about 100 or between about 100 and about 1,000 copies of the unit sequence. In some embodiments, the concatemeric region comprises at least about 10 copies of the unit sequence, at least about 20 copies of the unit sequence, at least about 30 copies of the unit sequence, at least about 40 copies of the unit sequence, or at least about 50 copies of the unit sequence. In some embodiments, the concatemeric region comprises at least about 20 copies of the unit sequence. In some embodiments, the concatemeric region comprises at least about 30 copies of the unit sequence.

In some embodiments, the concatemeric region is in the form of a nanoball, e.g., as generated using RCA. In some embodiments, the nanoball has a diameter of between about 0.1 μm and about 3 μm, e.g., between about 0.1 μm and about 0.5 μm (e.g., between about 0.2 μm and about 0.3 μm, or between about 0.3 μm and about 0.4 μm), between about 0.5 μm and about 1 μm, between about 0.8 μm and about 1.3 μm, or between about 1 μm and about 1.5 μm. In some embodiments, the nanoball has a diameter of between about 0.1 μm and about 3 μm, between about 0.1 μm and about 0.5 μm, between about 0.2 μm and about 0.3 μm, or between about 0.3 μm and about 0.4 μm.

In some embodiments, the concatemeric region is between about 1 and about 5 kilobases, between about 5 and about 10 kilobases, between about 10 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length, e.g., between about 45 and about 70 kilobases in length. In some embodiments, the concatemeric region is between about 1 and about 15 kilobases or between about 15 and about 25 kilobases in length.

In some embodiments, the detectable probe comprises a detection region capable of binding directly or indirectly to a detectably labeled oligonucleotide. In some embodiments, the unit sequence of the detectable probe comprises the detection region. In some embodiments, the provided methods further include contacting the biological sample with the detectably labeled oligonucleotide, thereby hybridizing the detectably labeled oligonucleotide to the detectable probe. In some embodiments, the provided methods involve detecting the detectably labeled oligonucleotide. In some embodiments, the provided methods involve detecting a complex with multiple molecules of the detectably labeled oligonucleotide hybridized to the detectable probe hybridized to the target nucleic acid.

In some embodiments, the unit sequence of the detectable probe is complementary to a circular or circularized oligonucleotide amplified by RCA to generate the concatemeric region of the detectable probe. In some embodiments, the unit sequence comprises one or more detectably labeled nucleotide residues. In some embodiments, the one or more detectably labeled nucleotide residues are incorporated during RCA into the detectable probe. In some embodiments, the unit sequence comprises a detection region capable of binding directly or indirectly to a detectably labeled oligonucleotide. In some embodiments, the detection region is or comprises all or a portion of the unit sequence. In some embodiments, the detection region is a portion of the unit sequence. In some embodiments, the unit sequence consists of the detection region. In some embodiments, the unit sequence is the detection region. In some embodiments, the unit sequence comprises the detection region and the target-binding region.

In some embodiments, the detectable probe comprises a single copy of the target-binding region. In some embodiments, the detectable probe comprises multiple copies of the target-binding region. In some embodiments, the detectable probe comprises copies of the detection region and copies of the target-binding region at a ratio of between about 1,000:1 and about 1:1 or between about 100:1 and about 1:1. In some embodiments, the unit sequence of the concatemeric region of the detectable probe comprises the target-binding region. In some embodiments, the concatemeric region comprises copies of the detection region and copies of the target-binding region at a ratio of between about 1,000:1 and about 1:1 or between about 100:1 and about 1:1.

In some embodiments, the detectable probe comprises a concatemeric region with multiple copies of detection region. In some embodiments, the concatemeric region comprises between about 10 and about 100, between about 50 and about 500, between about 100 and about 1,000, between about 1,000 and about 5,000, between about 5,000 and about 10,000, or more than 10,000 copies of the detection region. In some embodiments, the concatemeric region comprises between about 10 and about 100 or between about 100 and about 1,000 copies of the detection region. In some embodiments, the concatemeric region comprises at least about 10 copies of the detection region, at least about 20 copies of the detection region, at least about 30 copies of the detection region, at least about 40 copies of the detection region, or at least about 50 copies of the detection region. In some embodiments, the concatemeric region comprises at least about 20 copies of the detection region. In some embodiments, the concatemeric region comprises at least about 30 copies of the detection region.

In some embodiments, the concatemeric region of the detectable probe is an amplification product, e.g., any as described herein or generated according any of the methods described herein. In some embodiments, the concatemeric region of the detectable probe is an RCP, e.g., any as described herein or generated according any of the methods described herein. In some embodiments, the concatemeric region of the detectable probe is single stranded. In some embodiments, the concatemeric region of the detectable probe is a continuous single strand of nucleic acid. In some embodiments, the concatemeric region of the detectable probe is not comprised of multiple branched nucleic acid molecules.

In some embodiments, provided herein are steps of generating the detectable probe. In some embodiments, the provided methods include steps of performing RCA to generate the concatemeric region. In some embodiments, the concatemeric region is an RCP of a circular or circularized oligonucleotide. In some embodiments, the circular or circularized oligonucleotide comprises a sequence complementary to the detection region. In some embodiments, the circular or circularized oligonucleotide consists of the sequence complementary to the detection region. In some embodiments, the circular or circularized oligonucleotide comprises a sequence complementary to the target-binding region. In some embodiments, the generated detectable probes can be processed (e.g., purified, cleaved, processed to remove template, etc.) prior to being contacted with the biological sample.

In some embodiments, the detectable probe is generated by hybridizing the circular oligonucleotide or a circularizable oligonucleotide or oligonucleotide set to a binding oligonucleotide. In some embodiments, the circularized oligonucleotide is generated by circularizing the circularizable oligonucleotide or oligonucleotide set, e.g., by ligation. In some embodiments, a portion of the sequence of the circular or circularized oligonucleotide that is complementary to the detection region hybridizes to the binding oligonucleotide. In some embodiments, all of the sequence of the circular or circularized oligonucleotide that is complementary to the detection region hybridizes to the binding oligonucleotide. In some embodiments, the sequence of the circular or circularized oligonucleotide that is complementary to the detection region does not hybridize to the binding oligonucleotide.

In some embodiments, RCA is performed using the circular or circularized oligonucleotide as a template. In some embodiments, the RCA is primed by the binding oligonucleotide or a product thereof. In some embodiments, the binding oligonucleotide comprises a portion of the sequence of the target-binding region. In some embodiments, the binding oligonucleotide comprises the target-binding region. In some embodiments, the binding oligonucleotide or a product thereof is extended during RCA and remains a portion of the detectable probe following RCA. In some embodiments, the RCA for generating the RCP of the detectable probe is performed for less than about 15 minutes, less than about 30 minutes, less than about one hour, less than about two hours, or less than about three hours. In some embodiments, the RCA for generating the RCP of the detectable probe is performed for about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some embodiments, the RCP of the detectable probe is shorter than or about 0.5 bases, shorter than or about 1 kilobases, shorter than or about 1.5 kilobases, shorter than or about 2 kilobases, shorter than or about 2.5 kilobases, shorter than or about 3 kilobases, shorter than or about 3.5 kilobases, shorter than or about 4 kilobases, shorter than or about 4.5 kilobases, or shorter than or about 5 kilobases in length. In some embodiments, the RCP of the detectable probe is in the form of a nanoball having a diameter of less than or about 0.1 μm, less than or about 0.2 μm, less than or about 0.3 μm, less than or about 0.4 μm, less than or about 0.5 μm, less than or about 0.6 μm, less than or about 0.7 μm, less than or about 0.8 μm, less than or about 0.9 μm, or less than or about 1 μm.

FIGS. 1A-1B and 2A-2G depict exemplary oligonucleotide sets that include a binding oligonucleotide and a circular or circularizable oligonucleotide. These oligonucleotide sets can be used to generate detectable probes that are produced ex situ and that are pre-formed prior to their use in the in situ detection of a target nucleic acid. As shown in FIGS. 1A-1B, detectably labelled nucleotides can be incorporated into the detectable probe during RCA in which the circular or circularizable oligonucleotide is used as a template. Alternatively, in FIGS. 2A-2G, the circular or circularizable oligonucleotide can comprise a sequence that is complementary to the detection region. The circular or circularizable oligonucleotide can hybridize to the binding oligonucleotide, and if needed, a circularized oligonucleotide can be generated by ligation of the circularizable oligonucleotide. Next, an RCA reaction can be performed to generate a detectable probe (e.g., an RCP). In this reaction, the circular or circularized oligonucleotide can be used as a template, and the RCA reaction can be primed by the binding oligonucleotide or a product thereof. In some embodiments, the RCA generates a product that contains multiple copies of the detection region. In some instances, the generated detectable probe is isolated and purified prior to being contacted with a sample comprising the target nucleic acid.

In some embodiments as shown in FIGS. 1A-1B and 2A-2G, the binding oligonucleotide can comprise a target-binding region that will hybridize to a target sequence in the target nucleic acid. In FIGS. 1A, 2A, 2C, 2E, and 2G, the portion of the binding oligonucleotide that hybridizes to the circular or circularizable oligonucleotide does not include the target-binding region, leading to the production of a detectable probe that comprises a single copy of the target-binding region following RCA. In FIGS. 1B, 2B, 2E, and 2F, the portion of the binding oligonucleotide that hybridizes to the circular or circularizable oligonucleotide comprises the target-binding region, leading to the production of a detectable probe that comprises multiple copies of the target-binding region following RCA.

In some embodiments as shown in FIGS. 2A, 2B, and 2E-2G, the binding oligonucleotide can also comprise a region with all or a portion of the sequence of the generated detectable probe after RCA that can be detected (e.g., detection region), and this region of the binding oligonucleotide can hybridize to its complement in the circular or circularizable oligonucleotide. In some embodiments as shown in FIGS. 2E-2G, the binding oligonucleotide can comprise only a portion of the unit sequence of the generated detectable probe that can be detected (e.g., detection region), and the complement of the remainder of the detection region of the generated detectable probe can be included in a portion of the circular or circularizable oligonucleotide that does not hybridize to the binding oligonucleotide. Similarly, and as shown in FIG. 2F, the binding oligonucleotide can include only a portion of the sequence of the target-binding region of the generated detectable probe, and the complement of the remainder of the target-binding region of the generated detectable probe can be included in a portion of the circular or circularizable oligonucleotide that does not hybridize to the binding oligonucleotide. In some embodiments as shown in FIG. 2G, the detection region of the generated detectable probe can have a sequence complementary to the entirety of the circular or circularizable oligonucleotide. Alternatively, the binding oligonucleotide in some embodiments does not include a sequence of the detection region of the generated detectable probe, as shown in FIGS. 2C and 2D. The generated detectable probe can comprise a continuous concatemeric region with multiple copies of the unit sequence (e.g., detection region), with the circular or circularizable oligonucleotide used as a template, e.g., for RCA. A plurality of detectable probes each comprising a different target-binding region (and optionally a different detection region) for a specific target can be generated using a plurality of different circular or circularizable oligonucleotides.

In some embodiments, the generated detectable probe comprises modified nucleotides, such as detectably labeled nucleotides, incorporated into the concatemeric region of the detectable probe during RCA. In some embodiments, the detectably labeled nucleotides are fluorescently labeled. In some embodiments, the detectably labeled nucleotides are incorporated into one or more or all of the copies of the unit sequence in the detectable probe.

In some embodiments, the generated detectable probe comprises one or more optional sequences or sites, for instance, one or more additional detection regions so that the detectable probe comprises different detection regions for binding to different detectably labeled probes. For instance, as shown in FIG. 2H, the detectable probe can comprise a first detection region and a second detection region, where the first and second detection regions comprise different sequences. In some instance, different detectably labeled probes can be hybridized to the first and second detection regions in the same hybridization cycle (e.g., in the same hybridization mix) and detected in different detection channels (e.g., fluorescent color channels). In some instance, different detectably labeled probes can be hybridized to the first and second detection regions in separate hybridization cycles and detected in the separate cycles, using the same or different detection channels (e.g., fluorescent color channels). In some embodiments, each of the first and second detection regions independently comprises one or more barcode sequences. In some embodiments, two or more different barcode sequences in the detection region(s) are detected in the same hybridization cycle using different detection channels. In some embodiments, two or more different barcode sequences in the detection region(s) are detected in separate hybridization cycles, using the same or different detection channels.

In some embodiments, the generated detectable probe comprises one or more functional groups. In some embodiments, the functional groups are in the target-binding region and/or in the concatemeric region of the detectable probe. In some embodiments, the functional groups are in the binding oligonucleotide used as an RCA primer to generate the detectable probe. In some embodiments, the functional groups are in modified nucleotides incorporated into the concatemeric region of the detectable probe during RCA. In some embodiments, a modified nucleotide comprising a functional group is incorporated into one or more or all of the copies of the unit sequence in the detectable probe. In some embodiments, the detectable probe comprises one or more biotin or avidin/streptavidin moieties or derivatives or analogs of biotin or avidin/streptavidin. In some embodiments, the detectable probe comprises one or more biotin-modified nucleotide residues. In some embodiments, the detectable probes generated (e.g., in bulk solution) can be purified using a binding partner to the modified nucleotide residues. For example, immobilized streptavidin on a solid or stationary phase (e.g., on beads or resin in a column) can be used to bind to biotin moieties on the modified nucleotide residues and purify the detectable probes. In some embodiments, the detectable probe comprises one or more amine-modified nucleotide residues. In some embodiments, the amine-modified nucleotide comprises an acrylic acid N-hydroxysuccinimide moiety modification, a 5-Aminoallyl-dUTP moiety modification, a 5-Propargylamino-dCTP moiety modification, a N6-6-Aminohexyl-dATP moiety modification, and/or a 7-Deaza-7-Propargylamino-dATP moiety modification. In some embodiments, the modified nucleotides comprises base modifications, such as azide and/or alkyne base modifications, dibenzylcyclooctyl (DBCO) modifications, vinyl modifications, trans-Cyclooctene (TCO), and so on. Additional chemical modifications, including click chemistry functional groups, are described in Sections II.A and II.B and may be included in the detectable probes herein. In some embodiments, the detectable probe may comprise a crosslinking moiety. The biotin or avidin/streptavidin moieties, the amine-modifications, and click chemistry functional groups can be used in crosslinking, and additional crosslinking moieties are described in Sections II.A and II.B (in particular, Section II.A.vii) and may be included in the detectable probes herein.

### C. In Situ Detection and Analysis

In some embodiments, the detection of signals associated with the detectable probe may be spatial, e.g., in two or three dimensions. In some embodiments, the detection may be quantitative. In some embodiments, disclosed herein is a multiplexed assay where multiple target analytes (e.g., nucleic acids such as genes or RNA transcripts, or protein targets) are probed with multiple primary probes, and associated target nucleic acids are detected by sequential detection using one or more detectable probes and decoding of the signals associated with the one or more detectable probes. In some embodiments, detection of barcodes or subsequences of the barcode can occur in a cyclic manner (e.g., to generate a signal signature, also referred to as a signal code sequence).

In some embodiments, the provided method for analyzing target analytes is a multiplexed assay where multiple probes (e.g., primary probes) are used to detect multiple analytes simultaneously, e.g., using sequential probe hybridization and detection cycles. In some embodiments, one or more detections of one or more analytes (or associated nucleic acids) may occur sequentially. In some aspects, provided herein is a method for detecting the detectable probes, thereby generating a signal signature. In some instances, the signal signature corresponds to an analyte of a plurality of analytes in the sample. In some instances, the methods described herein are based, in part, on the development of a multiplexed biological assay and readout, in which a sample is first contacted with a plurality of nucleic acid probes, allowing the probes to directly or indirectly bind target analytes, which may then be optically detected (e.g., by detectable probes) in a temporally-sequential manner. In some instances, a plurality of probes or probe sets may be applied to a sample simultaneously. In some instances, a plurality of probes or probe sets may be applied to a sample sequentially. In some aspects, the method comprises sequential hybridization of detectable probes to create a spatiotemporal signal signature or code that identifies the analyte.

In some embodiments, a method disclosed herein comprises detecting one or more target analytes (e.g., cellular nucleic acid) in a sample using a plurality of primary probes configured to hybridize to the one or more target analytes, wherein each primary probe comprises (i) a target-hybridizing region configured to hybridize to a different target region in the corresponding target analytes, and (ii) a barcode region. In some embodiments, the sample is contacted with a plurality of detectable probes (e.g., each detectable probe comprising a target-binding region and a concatemeric region comprising multiple copies of a unit sequence), wherein each detectable probe is configured to directly or indirectly bind to a barcode sequence in the barcode regions of the plurality of primary probes. In some embodiments, the method further comprises detecting a signal associated with the plurality of detectable probes or absence thereof at one or more locations in the sample. In some embodiments, the sample is contacted with a subsequent plurality of detectable probes (e.g., each detectable probe comprising a target-binding region and a concatemeric region comprising multiple copies of a unit sequence, wherein each detectable probe in the subsequent plurality is configured to directly or indirectly bind to a subsequent barcode sequence in the barcode regions of the plurality of primary probes. In some embodiments, the method further comprises detecting a subsequent signal associated with the subsequent plurality of detectable probes or absence thereof at the one or more locations in the sample. In some embodiments, the method further comprises generating a signal code sequence comprising signal codes corresponding to the signal or absence thereof and the subsequent signal or absence thereof, respectively, at the one or more locations, wherein the signal code sequence corresponds to one of the one or more target analytes, thereby identifying the target analyte at the one or more locations in the sample. In some embodiments, multiple target analytes (e.g., cellular nucleic acids) are detected in the sample using sequential hybridization of the primary probes and/or the detectable probes (e.g., each detectable probe comprising a target-binding region and a concatemeric region comprising multiple copies of a unit sequence).

In some embodiments, the method comprises generating a signal code sequence at one or more locations in a sample, the signal code sequence comprising signal codes corresponding to the signals (or absence thereof) associated with detectable probes for in situ hybridization that are sequentially applied to the sample, wherein the signal code sequence corresponds to an analyte in the sample, thereby detecting the analyte at the one or more of the multiple locations in the sample.

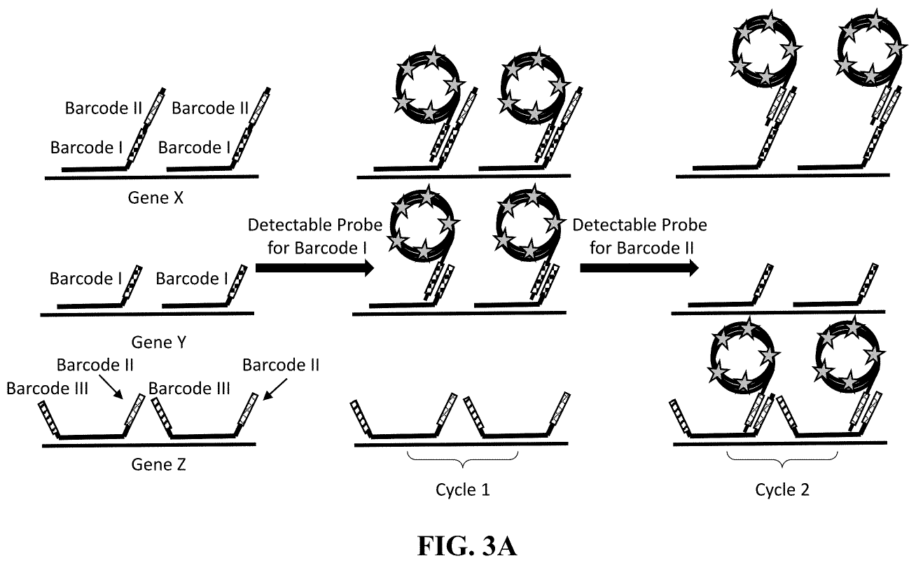

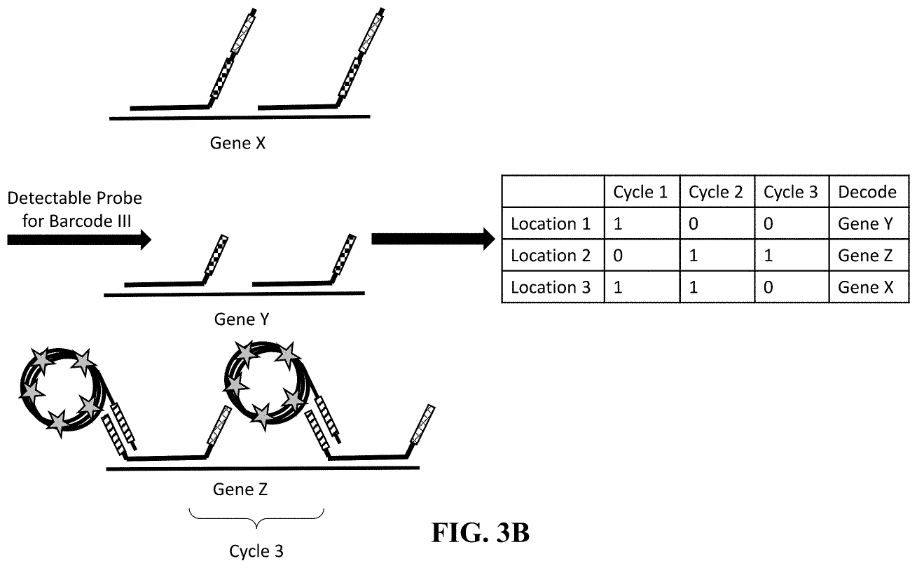

For instance, in FIGS. 3A-3B, each of Gene X, Gene Y, and Gene Z (e.g., RNA transcripts of the genes) can be targeted by a plurality of primary probes, each comprising a target-hybridizing region and a barcode region. The primary probes for Gene X can comprise Barcodes I and II, the primary probes for Gene Y can comprise Barcode I, and the primary probes for Gene Z can comprise Barcodes II and III. Barcodes I-III can be different, and the combinations of the barcodes in the primary probes can be used to encode the three genes. Signals associated with the barcodes at one or more locations in a sample can be detected in sequential probe hybridization cycles and used to decode the combinations of the barcodes, thereby decoding and detecting the three genes at the one or more locations in the sample. Detectable probes each comprising a target-binding region (binding to Barcode I, II, or III) and a concatemeric region (e.g., comprising multiple detection regions) can be used in sequential cycles to detect the signals associated with the barcodes in the primary probes. In some embodiments, because signals can be amplified using the concatemeric region, the number of primary probes needed to generate sufficient signals may be no more than 20, no more than 15, no more than 10, no more than 5, no more than 4, no more than 3, or no more than 2.

In some embodiments, a method disclosed herein comprises generating RCPs associated with one or more target analytes (e.g., cellular nucleic acid) in a sample. In some embodiments, the RCPs are detected in situ in a sample, thereby detecting the one or more target analytes. In some embodiments, each of the RCPs comprises multiple complementary copies of a barcode sequence, wherein the barcode sequence is associated with a target analyte in the sample and is assigned a signal code sequence. In some embodiments, the method comprises contacting the sample with a first detectable probe comprising (i) a recognition sequence (e.g., a target-binding region) complementary to a sequence in the complementary copies of the barcode sequence and (ii) a reporter such as a concatemeric region comprising multiple copies of a unit sequence. In some embodiments, the method comprises detecting a first signal or absence thereof from the reporter of the first detectable probe hybridized to its corresponding sequence of the complementary copies of the barcode sequence in the RCP, wherein the first signal or absence thereof corresponds to a first signal code in the signal code sequence. In some embodiments, the method comprises contacting the sample with a subsequent detectable probe comprising (i) a recognition sequence (e.g., a target-binding region) complementary to a sequence of the complementary copies of the barcode sequence and (ii) a reporter such as a concatemeric region comprising multiple copies of a unit sequence. In some embodiments, the method comprises detecting a subsequent signal or absence thereof from the reporter of the subsequent detectable probe hybridized to its corresponding sequence of the complementary copies of the barcode sequence in the RCP, wherein the subsequent signal or absence thereof corresponds to a subsequent signal code in the signal code sequence. In some embodiments, the signal code sequence comprising the first signal code and the subsequent signal code is determined at a location in the sample, thereby decoding the barcode sequence and identifying the target analyte at the location in the sample.

In some embodiments, the barcode sequence comprises one or more barcode positions each comprising one or more barcode subunits. In some embodiments, a barcode position in the barcode sequence partially overlaps an adjacent barcode position in the barcode sequence. In some embodiments, the first detectable probe and the subsequent detectable probe are in a set of detectable probes each comprising the same recognition sequence and a reporter. In some embodiments, the reporter of each detectable probe in the set comprises a binding site (e.g., a detection region) for a reporter probe comprising a detectable moiety (e.g., a detectably labeled oligonucleotide). In some embodiments, the reporter probe binding site (e.g., detection region) of the first detectable probe and the reporter probe binding site (e.g., detection region) of the subsequent detectable probe are the same. In some embodiments, the reporter probe binding site (e.g., detection region) of the first detectable probe and the reporter probe binding site (e.g., detection region) of the subsequent detectable probe are different. In some embodiments, the detectable moiety is a fluorophore and the signal code sequence is a fluorophore sequence uniquely assigned to the target analyte (e.g., cellular nucleic acid). In some embodiments, the detectable probes in the set are contacted with the sample sequentially in a pre-determined sequence which corresponds to the signal code sequence assigned to the barcode sequence. In some embodiments, the detectable probes in the set are contacted with the sample to determine signal codes in the signal code sequence until sufficient signal codes have been determined to decode the barcode sequence, thereby identifying the target analyte.

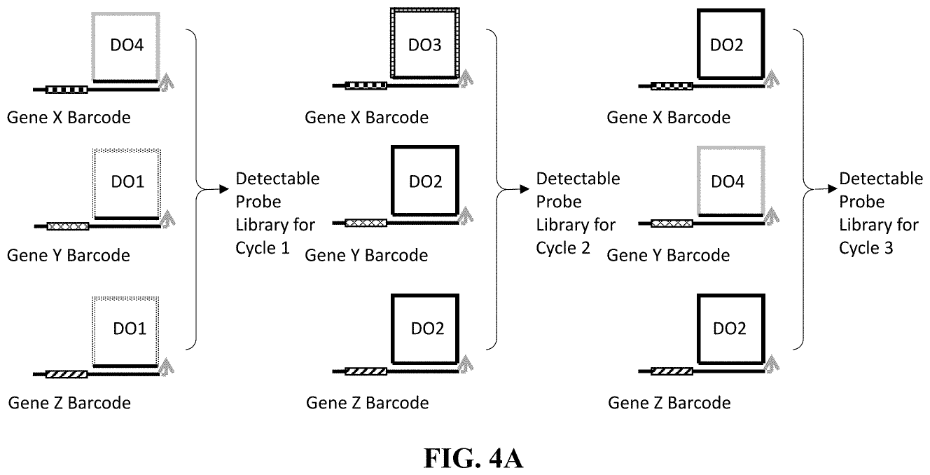

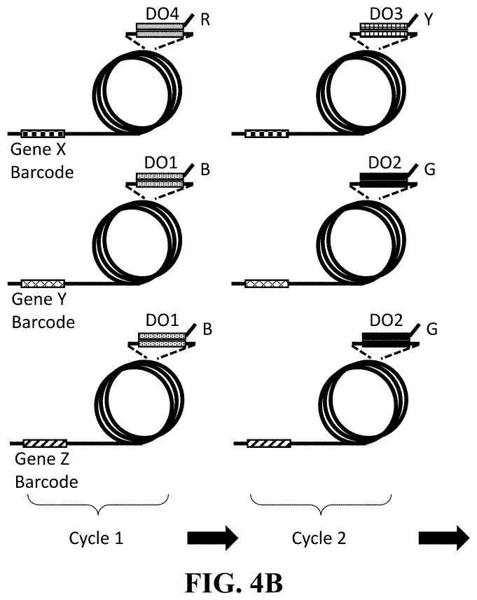

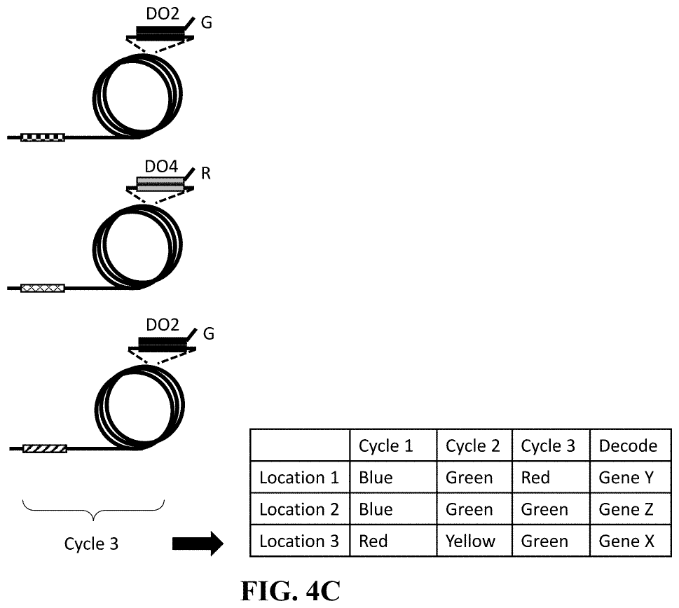

A plurality of detectable probes can be generated to provide a library of detectable probes, each comprising a target-binding region for a different analyte. Multiple libraries of detectable probes can be generated for use to combinatorially detect a plurality of analytes at one or more locations in a biological sample. For instance, in FIG. 4A, a first library of detectable probes (for Cycle 1) can be generated using binding oligonucleotides each comprising a barcode for a particular analyte, e.g., a barcode for Gene X, Gene Y, and Gene Z, respectively. Circular or circularizable oligonucleotides or oligonucleotide sets comprising different detection oligonucleotide (DO) sequences each complementary to a corresponding detection region can be contacted with the binding oligonucleotides for RCA. The circular or circularizable oligonucleotides or oligonucleotide sets may comprise a common sequence that hybridizes to the binding oligonucleotides, such that the binding oligonucleotides can prime the RCA reactions. The RCP for each gene may comprise a gene-specific barcode and multiple copies of the complement of a DO sequence. For example, in the first library of detectable probes (for Cycle 1), the detectable probe for Gene X can comprise the Gene X barcode and an RCP comprising complements of DO4; the detectable probe for Gene Y can comprise the Gene Y barcode and an RCP comprising complements of DO1; and the detectable probe for Gene Z can comprise the Gene Z barcode and an RCP comprising complements of DO1. Similarly, a second library of detectable probes (for Cycle 2) and a third library of detectable probes (for Cycle 3) can be generated, each comprising a barcode for Gene X, Gene Y, or Gene Z. The same DO sequence may be associated with different gene-specific barcodes in the same library of detectable probes. Between detectable probes in different libraries, the same gene-specific barcode can be associated with different DO sequences or the same DO sequence.

Each library of detectable probes can be contacted with a biological sample in a probe hybridization cycle, and the libraries of detectable probes can be applied in sequential probe hybridization cycles. Detectably labeled probes, e.g., fluorescently labeled Dos, can be hybridized to the detectable probes comprising complements of the DO sequences. Each DO sequence can correspond to a different “color,” for example, DO1 to blue (“B”), DO2 to green (“G”), DO3 to yellow (“Y”), and DO4 to red (“R”). FIGS. 4B-4C show that signals associated with the detectable probes (each of which may comprise a gene-specific barcode) at one or more locations in a sample can be detected in the sequential probe hybridization cycles, and the temporal combination or pattern of the “colors” can be detected and used to decode the gene-specific barcodes, thereby decoding and detecting the three genes at the one or more locations in the sample. The gene-specific barcode in each detectable probe can bind to a sequence (e.g., a complementary barcode sequence) in an RCP (e.g., as shown in FIG. 2H, left panel), a sequence in a primary probe (e.g., as shown in FIG. 2H, middle panel), or a sequence in an intermediate probe (e.g., as shown in FIG. 2H, right panel) which may be in a branched hybridization complex.

In some aspects, the provided methods involve analyzing, e.g., detecting or determining, one or more nucleic acid sequences. In some cases, the analysis is performed on one or more images captured, and may comprise processing the image(s) and/or quantifying signals observed. In some embodiments, the analysis comprises detecting a sequence (e.g., a barcode sequence) present in the sample. In some embodiments, the analysis comprises quantification of puncta (e.g., if amplification products are detected). In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further comprises displaying the information from the analysis or detection step. In some embodiments, software may be used to automate the processing, analysis, and/or display of data.

In some aspects, the provided methods involve analyzing, e.g., detecting or determining, one or more sequences associated with target analytes. In some embodiments, a primary probe hybridized to a cellular nucleic acid may comprise one or more target sequence(s) complementary to a target-binding region of the detectable probes provided herein. For example, a primary probe hybridized to a cellular nucleic acid may be detected.

In some aspects, the provided methods involve analyzing, e.g., detecting or determining, one or more sequences present in the polynucleotides and/or in a product or derivative thereof, such as in an amplified primary probe. In some embodiments, the primary probe or product thereof can be detected by providing detectable probes. In some embodiments, the methods comprise detecting all or a portion of the amplification product, such as one or more barcode sequences present in the amplification product. In some embodiments, the analysis and/or sequence determination comprises in situ hybridization to the amplification product or the probe(s).

In some examples, in situ hybridization of the pre-formed detectable probe to a target nucleic acid (e.g., RCP of probes targeting a cellular nucleic acid, or a probe binding directly or indirectly to a cellular nucleic acid) can be used for targeting an mRNA molecule. Exemplary oligonucleotides for generating a detectable probe including a concatemeric region with multiple copies of a unit sequence are shown in FIGS. 1A-1B and 2A-2G. In some embodiments, the detectable probe can be generated by an RCA reaction performed in vitro that incorporates detectably labelled nucleotides into the amplification product which comprises a concatemeric region (see, e.g., FIGS. 1A-1B). In some embodiments, the detectable probe comprises a plurality of internal detectably labeled nucleotides. FIG. 1C depicts in situ hybridization of the pre-formed detectable probe to a target nucleic acid with one or more target sequences that hybridize to the detectable probe. In some instances, the target nucleic acid may be a RCA-generated amplification product comprising multiple copies of a target sequence (left panel of FIG. 1C). In some instances, the target nucleic acid may be a primary probe comprising a region that is complementary to and hybridizes to the cellular nucleic acid and an overhang region comprising one or more target sequence(s) (middle panel of FIG. 1C). In some instances, the target nucleic acid may be an intermediate probe comprising a region that is complementary to and hybridizes to a primary probe; the primary probe comprises a region that is complementary to and hybridizes to the cellular nucleic acid; and the intermediate probe also comprises an overhang region comprising multiple copies of a target sequence (right panel of FIG. 1C).

In some instances, a target nucleic acid (e.g., a probe binding directly or indirectly to a cellular nucleic acid, or RCP of probes targeting a cellular nucleic acid) comprises at least one target sequence. In some instances, a target nucleic acid comprises one or more copies of the same target sequence. In some instances, a target nucleic acid comprises two or more different target sequences. In some instances, a target nucleic acid comprises one or more copies of each of the two or more different target sequences.

In some embodiments as shown in FIGS. 2A-2G, the detectable probe can be generated by an RCA reaction that amplifies a circular or circularized oligonucleotide that comprises a sequence complementary to a detection region. As shown in FIG. 2H, in situ hybridization of the pre-formed detectable probe can be used to hybridize to target nucleic acids. In some instances, the detectable probe can include multiple copies of sequences of the detection regions and can be detected with detectably labelled oligonucleotides that hybridize to a sequence of the detection region. In some instances, the target nucleic acid may be an RCA-generated amplification product comprising multiple copies of a target sequence (left panel of FIG. 2H). In some instances, the target nucleic acid may be a primary probe comprising a region that is complementary to and hybridizes to the cellular nucleic acid and an overhang region comprising one or more target sequence(s) (middle panel of FIG. 2H). In some instances, the target nucleic acid may be an intermediate probe comprising a region that is complementary to and hybridizes to a primary probe; the primary probe comprises a region that is complementary to and hybridizes to the cellular nucleic acid; and the intermediate probe also comprises an overhang region comprising one or more target sequence(s) (right panel of FIG. 2H). In some embodiments, each target sequence comprises a sequence complementary to a sequence of the target-binding region of the pre-formed detectable probe.

In some embodiments, the amplification of the primary probe is non-enzymatic.

In some embodiments, the detection or determination comprises hybridizing to the target nucleic acid, e.g., amplification product, a detectable probe provided herein comprising a concatemeric region comprising multiple copies of a unit sequence. In some embodiments, the detection or determination comprises imaging the target nucleic acid, e.g., amplification product.

In some embodiments, the in situ detection herein can comprise sequential hybridization, e.g., sequencing by hybridization and/or sequential in situ fluorescence hybridization. Sequential fluorescence hybridization can involve sequential hybridization of the detectable probes disclosed herein (e.g., each detectable probe comprising a target-binding region and a concatemeric region comprising multiple copies of a unit sequence). In some embodiments, a method disclosed herein comprises sequential hybridization of the detectable probes disclosed herein, including detectably labeled probes (e.g., concatemeric detectable probes comprising fluorescently labeled nucleotides incorporated therein during RCA) and/or detectable probes that are not detectably labeled per se but are capable of binding (e.g., via nucleic acid hybridization) and being detected by detectably labeled probes, such as concatemeric detectable probes comprising detection regions capable of hybridizing to fluorescently labeled probes. Exemplary methods comprising sequential fluorescence hybridization of detectable probes are described in U.S. Patent Publication Nos. 2019/0161796, 2020/0224244, 2022/0010358, 2021/0340618, and 2022/0064697, and International PCT Patent Publication No. WO 2021/138676, the entire contents of each of which are incorporated herein by reference.

Provided herein in some embodiments are methods involving the use of one or more probes (e.g., a detectable probe) for analyzing one or more target nucleic acid(s), such as a target nucleic acid (for example, associated with a messenger RNA) present in a cell or a biological sample, such as a tissue sample. In some embodiments, the target nucleic acid is associated with an mRNA in a tissue sample, and the detection or determination is performed when the target nucleic acid and/or the amplification product is in situ in the tissue sample. In some aspects, the provided embodiments can be employed for in situ detection of a target nucleic acid in a cell, e.g., in cells of a biological sample or a sample derived from a biological sample, such as a tissue section on a solid support, such as on a transparent slide.

In some aspects, provided herein are in situ assays using microscopy as a readout, e.g., hybridization, or other detection or determination methods involving an optical readout. In some aspects, detection or determination of a sequence of one, two, three, four, five, or more nucleotides of a target nucleic acid is performed in situ in a cell in an intact tissue. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCP or hybridization complex). In some embodiments, a method for spatially profiling analytes such as the transcriptome or a subset thereof in a biological sample is provided. In some embodiments, a provided method is quantitative and preserves the spatial information within a tissue sample without physically isolating cells or using homogenates. In some embodiments, the present disclosure provides methods for high-throughput profiling of one or more single nucleotides of interest in a large number of targets in situ, such as transcripts and/or DNA loci, and/or for detecting and/or quantifying nucleic acids in cells, tissues, organs, or organisms.

In some aspects, the provided methods comprise imaging the target nucleic acid, e.g., amplification product, via binding of the detectable probe and detecting the detectable label. In some embodiments, the detectable probe comprises a detectable label or binds a detectably labeled oligonucleotide that can be measured and quantitated. In some embodiments, the label, e.g., detectable label, comprises a directly or indirectly detectable moiety that is associated with (e.g., conjugated to) a molecule to be detected, e.g., comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, a fluorophore comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, Ypet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs, and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, and phycoerythrin.

Examples of bioluminescent markers comprise, but are not limited to, luciferase (e.g., bacterial, firefly and click beetle), luciferin, aequorin, and the like. Examples of enzyme systems having visually detectable signals comprise, but are not limited to, galactosidases, glucorimidases, phosphatases, peroxidases, and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), the entire contents of each of which are incorporated herein by reference. In some embodiments, exemplary techniques and methods methodologies applicable to the provided embodiments comprise those described in, for example, U.S. Pat. Nos. 4,757,141, 5,151,507 and 5,091,519, the entire contents of each of which are incorporated herein by reference. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), the entire contents of each of which are incorporated herein by reference. Labeling can also be carried out with quantum dots, as described in U.S. Pat. Nos. 6,322,901, 6,576,291, 6,423,551, 6,251,303, 6,319,426, 6,426,513, 6,444,143, 5,990,479, and 6,207,392, and US Patent Publication Nos. 2002/0045045 and 2003/0017264, the entire contents of each of which are incorporated herein by reference. In some aspects, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics, and energy transfer.

In some embodiments, the detectable probe comprises one or more detectably labelled, e.g., fluorescent, nucleotides. In some embodiments, the one or more detectably labelled nucleotides are incorporated into the concatemeric region of the detectable probe during generation of the detectable probe, e.g., during RCA. Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). For exemplary methods for custom synthesis of nucleotides having other fluorophores, see, Henegariu et al. (2000) Nature Biotechnol. 18:345, the entire contents of which are incorporated herein by reference.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (e.g., as described in Lakowicz et al. (2003) Bio Techniques 34:62, the entire contents of which are incorporated herein by reference).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some aspects, the antibody is an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, for instance with a hapten that is then bound by a capture agent, e.g., as disclosed in U.S. Pat. Nos. 5,344,757, 5,702,888, 5,354,657, 5,198,537, 4,849,336, and 5,073,562, the entire contents of each of which are incorporated herein by reference. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry, or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detectable probe and/or a detectably labeled oligonucleotide bound thereto. In some aspects, a fluorescence microscope is an optical microscope that uses fluorescence and phosphorescence instead of, or in addition to, reflection and absorption to study properties of organic or inorganic substances. In fluorescence microscopy, a sample can be illuminated with light of a wavelength which excites fluorescence in the sample. The fluoresced light, which is usually at a longer wavelength than the illumination, can then be imaged through a microscope objective. Two filters may be used in this technique: an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. In some aspects, the fluorescence microscope is any microscope that uses fluorescence to generate an image, whether it is a more simple set up like an epifluorescence microscope, or a more complicated design such as a confocal microscope, which can use optical sectioning to get better resolution of the fluorescent image.

In some embodiments, confocal microscopy is used for detection and imaging of the detectable probe and/or a detectably labeled oligonucleotide bound thereto. Confocal microscopy can use point illumination and a pinhole in an optically conjugate plane in front of the detector to eliminate out-of-focus signal. As only light produced by fluorescence very close to the focal plane can be detected, the image's optical resolution, particularly in the sample depth direction, can be much better than that of wide-field microscopes. However, as much of the light from sample fluorescence can be blocked at the pinhole, this increased resolution can be at the cost of decreased signal intensity—so long exposures may be required. As only one point in the sample is illuminated at a time, 2D or 3D imaging can require scanning over a regular raster (e.g., a rectangular pattern of parallel scanning lines) in the specimen. The achievable thickness of the focal plane can be defined mostly by the wavelength of the used light divided by the numerical aperture of the objective lens, but also by the optical properties of the specimen. The thin optical sectioning possible can make these types of microscopes particularly good at 3D imaging and surface profiling of samples. CLARITY™-optimized light sheet microscopy (COLM) provides an alternative microscopy for fast 3D imaging of large clarified samples. COLM can interrogate large immunostained tissues, permit increased speed of acquisition, and result in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed by sequential fluorescence hybridization (e.g., sequencing by hybridization). Sequential fluorescence hybridization can involve sequential hybridization of probes (detectable probes or detectably labeled oligonucleotide hybridized thereto) comprising an oligonucleotide and a detectable label.

In some embodiments, the barcodes are targeted by detectable probes or associated a detectably labeled oligonucleotides, such as fluorescently labeled oligonucleotides. In some embodiments, one or more decoding schemes are used to decode the signals, such as fluorescence, for sequence determination.

In some embodiments, nucleic acid hybridization can be used for sequencing. These methods can utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), the entire contents of each of which are incorporated herein by reference.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### IV. Kits

Also provided herein in some embodiments are kits containing any of the provided detectable probes. In some embodiments, the kits further include one or more polynucleotides, e.g., any described in Section III, for instance detectably labelled oligonucleotides that hybridize to the detection region of the detectable probes. In some embodiments, the kits further include reagents for performing the methods provided herein, for example reagents required for one or more steps comprising hybridization, ligation, amplification, detection, sequencing, and/or sample preparation as described herein, e.g., any as described in Section II. In some embodiments, the kit further comprises a target nucleic acid, e.g., a primary probe or an intermediate probe. In some embodiments, any or all of the polynucleotides are DNA molecules. In some embodiments, the target nucleic acid is a messenger RNA molecule. In some embodiments, the kit further comprises a ligase, for instance for forming a ligated circular probe from the circularizable probe or probe set. In some embodiments, the ligase has DNA-splinted DNA ligase activity. In some embodiments, the kit further comprises a polymerase, for instance for performing amplification of the circular or circularizable probe. In some embodiments, the polymerase is capable of using the ligated circular probe as a template for amplification. In some embodiments, the kit further comprises a primer for amplification.

In some embodiments, provided herein is a kit comprising a plurality of detectable probes, each comprising a barcode region as the target-binding region. The concatemeric region may comprise detectably labeled nucleotide residues, and/or detection regions capable of hybridizing to detectably labeled probes. In some embodiments, the barcode regions in the plurality of detectable probes are configured to combinatorially encode multiple analytes, for example, as shown in FIGS. 3A-3B, where the combinations of the presence or absence of Barcodes I-III in the primary probes can be used to encode the three genes.

In some embodiments, provided herein is a kit comprising binding oligonucleotides and circular or circularizable oligonucleotides or oligonucleotide sets for generating the library of detectable probes, e.g., as described in Section III-C. The binding oligonucleotides can each comprise a barcode for a particular analyte, e.g., a barcode for Gene X, Gene Y, and Gene Z, respectively, as depicted in FIG. 4A. The circular or circularizable oligonucleotides or oligonucleotide sets may comprise different detection oligonucleotide (DO) sequences.

In some embodiments, provided herein is a kit comprising a library of detectable probes, each comprising a target-binding region (e.g., a target-specific barcode) for a different analyte. In some embodiments, the target-specific barcode is used to identify the corresponding target analyte. In some embodiments, the target-specific barcode is a gene-specific barcode that can be used to identify a gene or a transcript thereof. In some embodiments, provided herein is a kit comprising multiple libraries of detectable probes, for use to combinatorially detect a plurality of analytes at one or more locations in a biological sample.

In some embodiments, the kit further comprises detectably labeled probes, e.g., fluorescently labeled detection oligonucleotides (Dos), which are configured to hybridize to the detectable probes comprising complements of the DO sequences. Each DO sequence can correspond to a different “color,” for example, DO1 to blue (“B”), DO2 to green (“G”), DO3 to yellow (“Y”), and DO4 to red (“R”), as shown in FIGS. 4B-4C. The target-specific barcode in each detectable probe can bind to a sequence (e.g., a complementary barcode sequence) in an RCA product (e.g., as shown in FIG. 2H, left panel), a sequence in a primary probe (e.g., as shown in FIG. 2H, middle panel), or a sequence in an intermediate probe (e.g., as shown in FIG. 2H, right panel) which may be in a branched hybridization complex. In some embodiments, the kit further comprises one or more circular or circularizable probes or probe sets for generating the RCA product, one or more primary probes, and/or one or more intermediate probes that form the branched hybridization complex.

The various components of the kit may be present in separate containers, or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as detectably labeled oligonucleotides or detectable labels. In some embodiments, the kits optionally contain other components, for example nucleic acid primers, enzymes, reagents, buffers, nucleotides, modified nucleotides, or reagents for additional assays.

### V. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein comprises (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range comprises one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample and/or a probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode comprises both a UMI and a spatial barcode. In some embodiments, a barcode comprises two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). One or more non-native bases can be included in a nucleic acid. Non-native bases and linkages that can be included in a nucleic acid or nucleotide are described, for example, in Ochoa and Milam, Molecules, 25(20):4659 (2020); and McKenzie et al., Chem Soc Rev., 50(8):5126-5164 (2021), the entire contents of each of which are incorporated herein by reference

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

Two nucleic acid sequences may become linked (e.g., hybridized) by an overlap of their respective terminal complementary nucleic acid sequences (e.g., 3′ termini). Such linking can be followed by primer extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence can be used as a template for single-strand synthesis of a corresponding cDNA molecule.

### (ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture comprises the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” comprises not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g., DNA polymerase activity of the wild-type sequence. Mutations can affect the activity profile of the enzymes, e.g., enhance or reduce the rate of polymerization, under different reaction conditions, e.g., temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer comprises a first universal sequence and/or the second primer comprises a second universal sequence.

In some embodiments, the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase, available from New England Biolabs, Ipswich, Mass.), and Ampligase™ (available from Epicentre Biotechnologies, Madison, Wis.). Derivatives, e.g., sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” comprises not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g., reverse transcriptase, activity of the wild-type sequence. The reverse transcriptase enzyme can be provided as part of a composition that comprises other components, e.g., stabilizing components that enhance or improve the activity of the reverse transcriptase enzyme, such as Rnase inhibitor(s), inhibitors of DNA-dependent DNA synthesis, e.g., actinomycin D. Many sequence-modified derivative or mutants of reverse transcriptase enzymes, e.g., M-MLV, and compositions including unmodified and modified enzymes are commercially available, e.g., ArrayScript™, MultiScribe™, ThermoScript™, and SuperScript® I, II, III, and IV enzymes.

Certain reverse transcriptase enzymes (e.g., Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g., an AMV or MMLV reverse transcriptase.

In some embodiments, the quantification of RNA and/or DNA is carried out by real-time PCR (also known as quantitative PCR or qPCR), such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)2 fragment, Fab′ fragment, Fab fragment, Fd fragment, or Fv fragment. In some embodiments, the term “antibody” comprises antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally comprises a detection method in which the existence or presence of the detectable label is confirmed, whereas quantifiable detection generally comprises a detection method having a quantifiable (e.g., numerically reportable) value such as an intensity, duration, polarization, and/or other properties.

In some embodiments, a plurality of detectable labels can be attached to a feature, probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (Di1C18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or comprises a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families may provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and—methoxy substituents, oxalates such as oxalyl active esters, p-nitrophenyl, N-alkyl acridinum esters, luciferins, lucigenins, or acridinium esters. In some embodiments, a detectable label is or comprises a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLES

The following examples are included for illustrative purposes only and are not intended to limit the scope of the present disclosure.

### Example 1: Design and Use of Detectable Probes with Detectably Labelled Concatemeric Regions

This Example describes the design and use of an oligonucleotide set for generating a detectable probe that can be used for the detection of a target nucleic acid. In this Example, the detectable probe has a detectably labelled concatemeric region, e.g., detectably labelled rolling circle amplification product (RCP), and permits highly sensitive detection of the target nucleic acid (e.g., an RCP that is generated in situ).

FIGS. 1A-1B depict exemplary oligonucleotide sets that include a binding oligonucleotide and a circular or circularizable oligonucleotide. These oligonucleotide sets can be used to generate detectable probes that are produced ex situ and that are pre-formed prior to their use in the in situ detection of a target nucleic acid. In FIGS. 1A-1B, the circular or circularizable oligonucleotide hybridizes to the binding oligonucleotide, and if needed, a circularized oligonucleotide is generated by ligation of the circularizable oligonucleotide. Next, a rolling circle amplification (RCA) reaction is performed to generate a detectable probe (e.g., an RCP). In this reaction, the circular or circularized oligonucleotide is used as a template, and the RCA reaction is primed by the binding oligonucleotide or a product thereof. During the RCA reaction, detectably labelled nucleotides, e.g., fluorescently labelled nucleotides, are incorporated into the RCP.

In FIGS. 1A-1B, the binding oligonucleotide comprises a target-binding region complementary to a target sequence in the target nucleic acid. In FIG. 1A, the portion of the binding oligonucleotide that hybridizes to the circular or circularizable oligonucleotide does not include the target-binding region. This can lead to the production of a detectable probe that comprises a single copy of the target-binding region following RCA. In FIG. 1B, the portion of the binding oligonucleotide that hybridizes to the circular or circularizable oligonucleotide comprises the target-binding region. This can lead to the production of a detectable probe that comprises multiple copies of the target-binding region following RCA. In FIGS. 1A-1B, the circular or circularizable oligonucleotide can also include additional sequences or sites, e.g., cleavage sites or purification moieties, to be amplified and incorporated into the RCP. The generated detectable probe comprises a continuous concatemeric region with multiple copies of the unit sequence of the circular or circularizable oligonucleotide used as a template. A plurality of detectable probes each comprising a different target-binding region for a specific target can be generated using a plurality of different circular or circularizable oligonucleotides. Various designs of the circular or circularizable oligonucleotide can be used, such as those as described in Example 2 below.

Following generation, the detectable probes are isolated then contacted with target nucleic acids in a biological sample. In one example, the detectable probes hybridize to target nucleic acids which are intermediate probes each including one or more copies of the target sequences that hybridize to the target-binding regions of the detectable probes (FIG. 1C, right). For example, a tissue section is incubated with a primary probe that hybridizes to a cellular nucleic acid, e.g., an mRNA, and the primary probe also comprises an overhang region that does not hybridize to the cellular nucleic acid. The tissue is then contacted with one or more intermediate probes each with a region for hybridizing to the overhang region of the primary probe (FIG. 1C, right), thereby forming a hybridization complex. In another example, the detectable probes hybridize to a target nucleic acid which is a primary probe bound to a cellular nucleic acid, thereby forming a hybridization complex (FIG. 1C, middle). The detectable probes that are generated in vitro using RCA are contacted with the sample and hybridize to the one or more probes bound (directly or indirectly) to the cellular nucleic acid, e.g., to the primary probe (FIG. 1C, middle) or the one or more intermediate probes hybridized to the primary probe (FIG. 1C, right). In some cases, the use of a detectable probe comprising the concatemeric region as described (e.g., either directly or indirectly labeled) can provide signal amplification and allow for a decrease in the number of primary probes needed for signal detection. In some aspects, the detectable probe can provide a compact signal for detection and decoding. Images are acquired using a microscope to detect the pre-formed detectable probes with detectably labeled nucleotides incorporated that are bound to target sequences contained in the hybridization complexes.

In some cases, a tissue section is incubated with a probe set mixture that hybridizes to a cellular nucleic acid, e.g., an mRNA, in order to form an RCP in situ (FIG. 1C, left). The RCP generated in situ comprises multiple copies of the target sequence that will hybridize to the target-binding regions of the detectable probes. For RCP generation, a probe set mixture containing padlock probes is heated, cooled down to room temperature, and incubated with a thin tissue section sample and hybridization buffer for hybridization of the probe sets to mRNAs in the sample. The sample is then washed and incubated at room temperature with a T4 DNA ligase for ligation of the 5′ and 3′ ends of the padlock probes to form circular probes. A stringent wash is then performed, and the sample is then incubated with an RCA mixture containing a Phi29 DNA polymerase and dNTP at approximately 30° C. for RCA of the circular probes. In some instances, modified nucleotide bases (e.g., 5-(3-aminoallyl)-dUTP) are also comprised in the RCA mixture. Following RCA, the sample is contacted with the pre-formed detectable probes to hybridize the detectable probes to the target sequences contained in the RCP generated in situ, as shown in the left panel of FIG. 1C. Images are acquired using a confocal microscope to detect the bound detectable probes with incorporated detectably labeled nucleotides bound to the generated RCP associated with the cellular nucleic acid.

### Example 2: Design and Use of Detectable Probes Having Concatemeric Regions with Multiple Copies of a Detection Region

This Example describes the design and use of an oligonucleotide set for generating a detectable probe that can be used for the detection of a target nucleic acid. In this Example, the detectable probe has a concatemeric region that has multiple copies of a detection region that can hybridize to a detectably labelled oligonucleotide. The detectable probe permits highly sensitive detection of the target nucleic acid. In some cases, the target nucleic acid is a rolling circle amplification product (RCP) that is generated in situ. In some cases, the target nucleic acid is comprised by a primary probe or an intermediate probe that binds directly or indirectly to an analyte (e.g., a cellular nucleic acid such as an mRNA) in the biological sample.

FIGS. 2A-2G depict exemplary oligonucleotide sets that include a binding oligonucleotide and a circular or circularizable oligonucleotide. These oligonucleotide sets can be used to generate detectable probes that are produced ex situ and that are pre-formed prior to their use in the in situ detection of a target nucleic acid. In FIGS. 2A-2G, the circular or circularizable oligonucleotide comprises a sequence that is complementary to the detection region. The circular or circularizable oligonucleotide hybridizes to the binding oligonucleotide, and if needed, a circularized oligonucleotide is generated by ligation of the circularizable oligonucleotide. Next, a rolling circle amplification (RCA) reaction is performed to generate a detectable probe (e.g., an RCP). In this reaction, the circular or circularized oligonucleotide is used as a template, and the RCA reaction is primed by the binding oligonucleotide or a product thereof. As a result of the RCA reaction, the ensuing RCP contains multiple copies of the detection region. In some aspects, the generation of the detectable probes in solution can allow for a fast and controllable reaction to generate detectable probes described (e.g., either directly or indirectly labeled) that can provide signal amplification.

In FIGS. 2A-2G, the binding oligonucleotide comprises a target-binding region that is complementary to a target sequence in the target nucleic acid. In FIGS. 2A, 2C, 2E, and 2G, the portion of the binding oligonucleotide that hybridizes to the circular or circularizable oligonucleotide does not include the target-binding region. This can lead to the production of a detectable probe that comprises a single copy of the target-binding region following RCA. In FIGS. 2B, 2E, and 2F, the portion of the binding oligonucleotide that hybridizes to the circular or circularizable oligonucleotide comprises the target-binding region. This can lead to the production of a detectable probe that comprises multiple copies of the target-binding region following RCA.

In FIGS. 2A, 2B, and 2E-2G, the binding oligonucleotide also comprises a region with all or a portion of the sequence of detectable probe to be generated after RCA that can be detected (e.g., detection region), and this region of the binding oligonucleotide hybridizes to its complement in the circular or circularizable oligonucleotide. In FIGS. 2E-2G, the binding oligonucleotide comprises only a portion of the sequence of detectable probe to be generated that can be detected (e.g., detection region), and the complement of the remainder of the detection region of the detectable probe to be generated is included in a portion of the circular or circularizable oligonucleotide that does not hybridize to the binding oligonucleotide. Similarly, and as shown in FIG. 2F, the binding oligonucleotide can include only a portion of the sequence of the target-binding region of the detectable probe to be generated, and the complement of the remainder of the target-binding region of the detectable probe to be generated is included in a portion of the circular or circularizable oligonucleotide that does not hybridize to the binding oligonucleotide. In FIG. 2G, the detection region of the detectable probe to be generated has a sequence complementary to the entirety of the circular or circularizable oligonucleotide. Alternatively, the binding oligonucleotide does not include a sequence of the detection region of the detectable probe to be generated, as shown in FIGS. 2C and 2D. The detectable probe to be generated comprises a continuous concatemeric region with multiple copies of the unit sequence (e.g., detection region) of the circular or circularizable oligonucleotide used as a template. A plurality of detectable probes each comprising a different target-binding region (and optionally different detection region) for a specific target can be generated using a plurality of different circular or circularizable oligonucleotides, e.g., as shown in FIG. 4A.

Following their generation, the detectable probes are isolated then hybridized to target nucleic acids in a biological sample. In one example, the detectable probes hybridize to target nucleic acids which are intermediate probes bound (directly or indirectly) to a cellular nucleic acid (FIG. 2H, right). In one example, a tissue section is incubated with a primary probe that hybridizes to a cellular nucleic acid, e.g., an mRNA, in order to form a complex in situ. The primary probe also comprises a overhang region that does not hybridize to the cellular nucleic acid. The tissue is then contacted with one or more intermediate probes each with a region for hybridizing to the overhang region of the primary probe (FIG. 2H, right) and each intermediate probe comprises one or more copies of the target sequence that will hybridize to the target-binding regions of the detectable probes. The sample is contacted with the pre-formed detectable probes to hybridize the detectable probes to the target sequences contained in the intermediate probes of the complexes (primary probe-intermediate probe complex) formed in situ. After hybridization of the detectable probes, the sample is contacted with detectably labelled oligonucleotides that hybridize to the detection regions of the detectable probes, as shown in the right panel of FIG. 2H. In some instances, the detectable probes hybridize to target nucleic acids which are primary probes bound directly to a cellular nucleic acid (FIG. 2H, middle). The primary probe has a region for hybridizing to the cellular nucleic acid and an overhang region that comprises one or more copies of the target sequence that can hybridize to the target-binding regions of the detectable probes.

In another example, a tissue section is incubated with a probe set mixture that hybridizes to a cellular nucleic acid, e.g., an mRNA, in order to form an RCP in situ. The RCP generated in situ comprises multiple copies of the target sequence that will hybridize to the target-binding regions of the detectable probes. A probe set mixture containing padlock probes is heated, cooled down to room temperature, and incubated with a thin tissue section sample and hybridization buffer for hybridization of the probe sets to mRNAs in the sample. The sample is then washed and incubated at room temperature with a T4 DNA ligase for ligation of the 5′ and 3′ ends of the padlock probes to form circular probes. A stringent wash is then performed. After the stringent wash, the sample is then incubated with an RCA mixture containing a Phi29 DNA polymerase and dNTP at approximately 30° C. for RCA of the circular probes. In some instances, modified nucleotide bases (e.g., 5-(3-aminoallyl)-dUTP) are also comprised in the RCA mixture. Following RCA, the sample is contacted with the pre-formed detectable probes to hybridize the detectable probes to the target sequences contained in the RCP generated in situ. After hybridization of the detectable probes, the sample is contacted with detectably labelled oligonucleotides that hybridize to the detection regions of the detectable probes, as shown in the left panel of FIG. 2H. Images are acquired using a confocal microscope.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
(a) contacting the biological sample with a pre-formed detectable probe comprising (i) a target-binding region that hybridizes to a target sequence and (ii) a concatemeric region comprising at least 20 copies of a unit sequence,
wherein the biological sample comprises a target nucleic acid comprising the target sequence; and
(b) detecting a signal associated with the detectable probe, thereby detecting the target nucleic acid or a sequence thereof in the biological sample.

2. The method of claim 1, wherein the concatemeric region of the pre-formed detectable probe is a rolling circle amplification (RCA) product generated prior to the contacting in step (a).

3. A method for analyzing a biological sample, comprising:
(a) contacting the biological sample with a detectable probe,
wherein the biological sample comprises a target nucleic acid comprising a target sequence, and
wherein the detectable probe comprises (i) a target-binding region that hybridizes to the target sequence and (ii) a concatemeric region comprising multiple copies of a unit sequence, wherein the concatemeric region is a rolling circle amplification (RCA) product generated prior to contacting the biological sample; and
(b) detecting a signal associated with the detectable probe, thereby detecting the target nucleic acid or a sequence thereof in the biological sample.

4. (canceled)

5. The method of claim 1, wherein the detectable probe comprises one or more detectably labelled nucleotides.

6-7. (canceled)

8. The method of claim 1, wherein the unit sequence comprises a detection region capable of binding directly or indirectly to a detectably labeled oligonucleotide.

9-12. (canceled)

13. The method of claim 8, further comprising before the detecting in step (b), hybridizing the detectably labeled oligonucleotide to the detectable probe or to an intermediate probe that hybridizes to the detectable probe, thereby indirectly hybridizing the detectably labeled oligonucleotide to the detectable probe, wherein the detecting in step (b) comprises detecting the detectably labeled oligonucleotide.

14-17. (canceled)

18. The method of claim 1, wherein the detecting in step (b) is performed in situ in the biological sample.

19-22. (canceled)

23. The method of claim 1, wherein the target-binding region is within the unit sequence of the concatemeric region.

24-25. (canceled)

26. The method of claim 1, wherein the detectable probe comprises one or more cleavage sites.

27-31. (canceled)

32. The method of claim 1, wherein the detectable probe is generated prior to the contacting in step (a) by:
(i) hybridizing a circular oligonucleotide or a circularizable oligonucleotide or oligonucleotide set to a binding oligonucleotide, and
(ii) performing RCA using the circular oligonucleotide or a circularized oligonucleotide as template, wherein the circularized oligonucleotide is generated by circularizing the circularizable oligonucleotide or oligonucleotide set, and wherein the RCA is primed by the binding oligonucleotide or a product thereof.

33-41. (canceled)

42. The method of claim 1, wherein the concatemeric region is in the form of a nanoball having a diameter of between about 0.1 μm and about 1.5 μm.

43-44. (canceled)

45. The method of claim 1, wherein the target nucleic acid comprises or is a cellular nucleic acid molecule or a reporter oligonucleotide of a labeling agent, the labeling agent comprising an analyte-binding region and the reporter oligonucleotide.

46. (canceled)

47. The method of claim 1, wherein the target nucleic acid comprises or is a primary probe that hybridizes to a cellular nucleic acid molecule.

48. (canceled)

49. The method of claim 47, wherein the primary probe comprises one or more barcode sequences, and the target-binding region of the detectable probe hybridizes to the one or more barcode sequences in the primary probe.

50-55. (canceled)

56. The method of claim 1, wherein the target nucleic acid comprises an RCA product of a circular or circularized probe that hybridizes to a nucleic acid molecule in the biological sample, wherein the circular or circularized probe comprises a barcode region.

57. The method of claim 56, further comprising generating the target nucleic acid in situ in the biological sample.

58-67. (canceled)

68. The method of claim 1, wherein the method comprises imaging the biological sample to detect the detectable probe.

69-72. (canceled)

73. The method of claim 1, wherein the biological sample is a tissue sample.

74-84. (canceled)

85. A detectable probe comprising (i) a target-binding region that hybridizes to a target sequence and (ii) a concatemeric region comprising multiple copies of a unit sequence, wherein the concatemeric region is a rolling circle amplification (RCA) product.

86. The detectable probe of claim 85, wherein the unit sequence comprises a detectable label or a detection region capable of binding directly or indirectly to a detectably labeled oligonucleotide.

87-97. (canceled)
